(19) United States
(12) Patent Application Publication
(10) Pub. No.: US 2024/0301483 A1
(43) Pub. Date: Sep. 12, 2024

(54) DNA MUTATION DETECTION EMPLOYING ENRICHMENT OF MUTANT POLYNUCLEOTIDE SEQUENCES AND MINIMALLY INVASIVE SAMPLING

(71) Applicants: Michael J Powell, Alamo, CA (US); Aiguo Zhang, San Ramon, CA (US)

(72) Inventors: Michael J Powell, Alamo, CA (US); Aiguo Zhang, San Ramon, CA (US)

(21) Appl. No.: 18/655,178

(22) Filed: May 3, 2024

Related U.S. Application Data
(63) Continuation of application No. 18/348,306, filed on Jul. 6, 2023, now abandoned.
Application No. 18/348,306 is a continuation of application No. 18/051,007, filed on Oct. 30, 2022, now abandoned.
Application No. 18/051,007 is a continuation of application No. 17/703,874, filed on Mar. 24, 2022, now abandoned.
Application No. 17/703,874 is a continuation of application No. 17/395,403, filed on Aug. 5, 2021, now abandoned.
Application No. 17/395,403 is a continuation of application No. 17/135,714, filed on Dec. 28, 2020, now abandoned.
Application No. 17/135,714 is a continuation of application No. 16/845,888, filed on Apr. 10, 2020, now abandoned.
Application No. 16/845,888 is a continuation of application No. 16/559,226, filed on Sep. 3, 2019, now abandoned.
Application No. 16/559,226 is a continuation of application No. 14/822,874, filed on Aug. 10, 2015, now Pat. No. 10,400,277.
(60) Provisional application No. 62/010,339, filed on Jun. 10, 2014.
(60) Provisional application No. 62/010,357, filed on Jun. 10, 2014.
(60) Provisional application No. 62/010,359, filed on Jun. 10, 2014.

(51) Int. Cl. C12Q 1/6858

(52) U.S. Cl. CPC C12Q 1/6858

(57) ABSTRACT

The invention relates to a method for enriching a target polynucleotide sequence containing a genetic variation said method comprising: (a) providing two primers targeted to said target polynucleotide sequence; (b) providing a target specific xenonucleic acid clamp oligomer specific for a wildtype polynucleotide sequence; (c) generating multiple amplicons using PCR under specific temperature cycling conditions; and (d) detecting said amplicons.

This application is a continuation of U.S. Ser. No. 18/348,306 filed Jul. 6, 2023; which application is a continuation of U.S. Ser. No. 18/051,007 filed Oct. 30, 2022; which application is a continuation of U.S. Ser. No. 17/703,874 filed Mar. 24, 2022; which application is a continuation of U.S. Ser. No. 17/395,403 filed Aug. 5, 2021; which application is a continuation of U.S. Ser. No. 17/135,714 filed Dec. 28, 2020; which application is a continuation of U.S. Ser. No. 16/845,888 filed Apr. 10, 2020, now abandoned; which application is a continuation of U.S. Ser. No. 16/559,226 filed Sep. 3, 2019, now abandoned; which application is a continuation of U.S. Ser. No. 14/822,874 filed Aug. 10, 2015; now U.S. Pat. No. 10,400,277 issued Sep. 3, 2019; the contents of which are incorporated herein in their entirety. This application also claims the priority benefit under 35 U.S.C. section 119 of U.S. Provisional Patent Application No. 62/010,339 entitled “Method For Enrichment Of Target Mutant Polynucleotide Sequences” filed on Jun. 10, 2014; U.S. Provisional Patent Application No. 62/010,357 entitled “Detection Of Multiple Mutations In A Single Tube Using Qclamp Assay Qclamp Mplex” filed on Jun. 10, 2014; and U.S. Provisional Patent Application No. 62/010,359 entitled “Liquid Biopsy” filed on Jun. 10, 2014; which are in their entirety herein incorporated by reference.

### FIELD OF THE INVENTION

The present invention relates to DNA mutation detection. The invention further relates to enrichment of mutant polynucleotide sequences. The present invention further relates to minimally invasive sampling and analysis of mutations in clinical samples.

The instant invention also relates to a method for determining whether a target polynucleotide sequence contained in a nucleic acid sample has nucleotide variation(s) in a selected region thereof, the steps of which involve the use of a pair of primers that allows the formation of a PCR product having a sequence covering that of the selected region of the target polynucleotide sequence via a PCR process, and a peptide nucleic acid (PNA) that acts as a PCR clamp as well as a sensor probe. This invention also relates to a kit for use in determining the presence of nucleotide variation(s) in the target polynucleotide sequence, which comprises the pair of primers and the PNA.

The present embodiments relate to precision molecular diagnostics, and in particular, to compositions in detecting sequence variants, such as SNPs, insertions deletions, and altered methylation patterns, from samples. The embodiments disclosed herein can be used to detect (and quantify) sequence variants present in samples that include an excess of wild-type sequences.

### BACKGROUND OF THE INVENTION

Polymerase chain reaction (PCR) is a widely used technique for the detection of pathogens. The technique uses a DNA polymerase used to amplify a piece of DNA by in vitro enzymatic replication. The PCR process generates DNA that is used as a template for replication. This results in a chain reaction that exponentially amplifies the DNA template.

Technologies for genomic detection most commonly use DNA probes to hybridize to target sequences. To achieve required sensitivity, the use of PCR to amplify target sequences has remained standard practice in many labs. While PCR has been the principle method to identify genes associated with disease states, the method has remained confined to use within a laboratory environment. Most current diagnostic applications that can be used outside of the laboratory are based on antibody recognition of protein targets and use ELISA-based technologies to signal the presence of a disease. These methods are fast and fairly robust, but they can lack the specificity associated with nucleic acid detection.

With the advent of molecular diagnostics and the discovery of numerous nucleic acid biomarkers useful in the diagnosis and treatment of conditions and diseases, detection of nucleic acid sequences, and sequence variants, mutations and polymorphisms has become increasingly important. In many instances, it is desirable to detect sequence variants or mutations (which may in some instances, differ by one a single nucleotide) present in low copy numbers against a high background of wild-type sequences. For example, as more and more somatic mutations are shown to be biomarkers for cancer prognosis and prediction of therapeutic efficacy, the need for efficient and effective methods to detect rare mutations in a sample is becoming more and more critical. In the case in which one or more allelic variants is/are present in low copy number compared to wild-type sequences, the presence of excess wild-type target sequence creates challenges to the detection of the less abundant variant target sequence. Nucleic acid amplification/detection reactions almost always are performed using limiting amounts of reagents. A large excess of wild-type target sequences, thus competes for and consumes limiting reagents. As a result amplification and/or detection of rare mutant or variant alleles under these conditions is substantially suppressed, and the methods may not be sensitive enough to detect the rare variants or mutants. Various methods to overcome this problem have been attempted. These methods are not ideal, however, because they either require the use of a unique primer for each allele, or the performance of an intricate melt-curve analysis. Both of these shortcomings limit the ability and feasibility of multiplex detection of multiple variant alleles from a single sample.

### BRIEF DESCRIPTION OF THE FIGURES

FIG. 1 illustrates the mechanism of the XNA clamping process.

FIG. 2 shows the differential melting temperature (Tm) between the XNA clamp bound to mutant templates vs wild type templates.

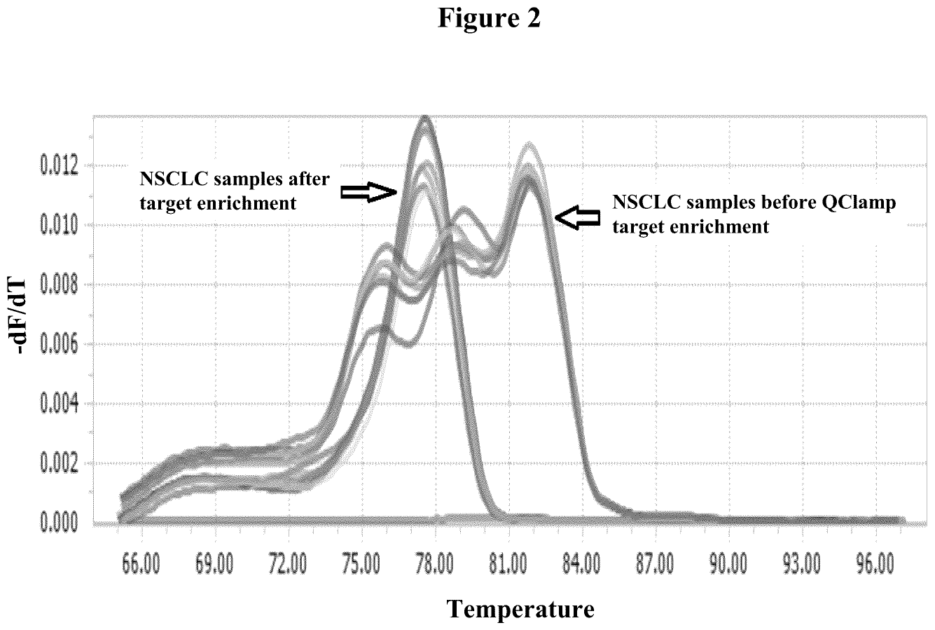

FIG. 3 show specific hydrolysis probe having a different fluorophore (and quencher) selected from the available fluorophores for multiplex applications.

FIG. 4 is a representative fluorophore spectral data and quencher selection guide.

FIG. 5 shows a specific locus specific hydrolysis probe assay.

FIG. 6 is a schematic illustrating how circulating tumor cells (CTC's) and cell-free DNA (cfDNA) derived from tumor cells are present in the peripheral blood of cancer patients.

### REFERENCE TO SEQUENCE LISTING

The present application is being filed along with a Sequence Listing in electronic format. The Sequence Listing is provided as a file entitled “DiaC9012.xml”, created May 3, 2024, which is 208,507 bytes in size. The information in the electronic format of the Sequence Listing is incorporated herein by reference in its entirety.

### SUMMARY OF THE INVENTION

Detection of rare sequence variants in biological samples presents numerous challenges. The methods and kits disclosed herein provide for improved, efficient means to detect rare mutations within a high background of wild-type allelic sequences using real-time amplification methods.

The instant invention provides a method for enriching a target polynucleotide sequence containing a genetic variation said method comprising: (a) providing two primers targeted to said target polynucleotide sequence; (b) providing a target specific xenonucleic acid clamp oligomer specific for a wildtype polynucleotide sequence; (c) generating multiple amplicons using PCR under specific temperature cycling conditions; and (d) detecting said amplicons.

The invention also relates to a method for enriching multiple target polynucleotide sequences containing a genetic variation said method comprising: (a) providing a library of amplifying primers targeted to said multiple target polynucleotide sequence; (b) providing a library of target specific xenonucleic acid clamp oligomer specific for multiple wildtype polynucleotide sequences; (c) generating multiple amplicons using PCR under specific temperature cycling conditions; and (d) detecting said amplicons.

The invention further relates to a method for conducting a minimally invasive biopsy in a mammalian subject suspected of a having a neoplastic disease, said method comprising: (a) sampling of target polynucleotides derived from said mammalian subject; (b) providing a library of amplifying primers targeted to said multiple target poly-nucleotide sequence; (c) providing a library of target specific xenonucleic acid clamp oligomer specific for multiple wildtype polynucleotide sequences; (d) generating multiple amplicons using PCR under specific temperature cycling conditions; and (e) detecting said amplicons.

The invention is also directed to means and methodology for the rapid isolation of genetic material from biological fluids and the sensitive detection of somatic and germ-line mutations present in circulating cells and cell-free genetic material obtained from said biological fluids using gene amplification and xeno-nucleic acid (XNA) clamping.

This invention provides a method for determining whether a target polynucleotide sequence contained in a nucleic acid sample has nucleotide variation(s) in a selected region thereof, comprising the steps of: providing a pair of a first primer and a second primer which allows the formation of a PCR product having a sequence covering that of the selected region of the target polynucleotide sequence via a PCR process, the first primer having a sequence identical to that of a first region located upstream of the selected region of the target polynucleotide sequence, the second primer having a sequence based on that of a second region located downstream of the selected region of the target polynucleotide sequence, wherein the 6′-end of the sequence of the first region is spaced apart from the 5′-end of the sequence of the sequence of the selected region by 30 nucleotides or more;
providing a detectable peptide nucleic acid probe having a sequence that complements fully the sequence of the selected region of the target polynucleotide sequence having no nucleotide variation(s) therein, such that hybridization of the detectable peptide nucleic acid probe to the selected region of the target polynucleotide sequence having no nucleotide variation(s) results in the formation of a duplex having a melting temperature;
determining the melting temperature of the duplex;
admixing the detectable peptide nucleic acid probe and the pair of the first primer and the second primer with the nucleic acid sample to form a mixture;
subjecting the mixture to a PCR process including an extension reaction set to run at a temperature lower than the melting temperature of the duplex by 5 to 20° C., such that a mixture of PCR products is obtained; and
subjecting the mixture of PCR products thus-obtained to a melting analysis to determine melting temperatures of the PCR products, wherein the presence of at least one melting temperature lower than the melting temperature of the duplex is indicative of the nucleotide variation(s) in the selected region of the target polynucleotide sequence contained in the nucleic acid sample.

The invention also provides a kit for determining whether a target polynucleotide sequence contained in a nucleic acid sample has nucleotide variation(s) in a selected region thereof, comprising: a detectable peptide nucleic acid probe having a sequence that complements fully the sequence of the selected region of the target polynucleotide sequence having no nucleotide variation(s) therein, such that hybridization of the detectable peptide nucleic acid probe to the selected region of the target polynucleotide sequence having no nucleotide variation(s) results in the formation of a duplex having a melting temperature;

a pair of a first primer and a second primer which allows the formation of a PCR product having a sequence covering that of the selected region of the target polynucleotide sequence via a PCR process, the first primer having a sequence identical to that of a first region located upstream of the selected region of the target polynucleotide sequence, the second primer having a sequence based on that of a second region located downstream of the selected region of the target polynucleotide sequence, wherein the 5′-end of the sequence of the first region is spaced apart from the 5′-end of the sequence of the sequence of the selected region by 30 nucleotides or more; and an instruction sheet providing guidance for a user to use the detectable peptide nucleic acid probe and the pair of the first primer and the second primer in a method as described above.

### REFERENCE TO SEQUENCE LISTING

The present application is being filed along with a Sequence Listing in electronic format. The Sequence Listing is provided as a file entitled “DiaC9012.xml”, created May 3, 2024, which is 208,507 bytes in size. The information in the electronic format of the Sequence Listing is incorporated herein by reference in its entirety.

### DETAILED DESCRIPTION OF THE INVENTION

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. Use of “or” means “and/or” unless stated otherwise. The term “and/or” means that the terms before and after can be taken together or separately. For illustration purposes, but not as a limitation, “X and/or Y” can mean “X” or “Y” or “X and Y”. Whenever a range of values is provided herein, the range is meant to include the starting value and the ending value and any value or value range there between unless otherwise specifically stated. For example, “from 0.2 to 0.5” means 0.2, 0.3, 0.4, 0.5; ranges there between such as 0.2-0.3, 0.3-0.4, 0.2-0.4; increments there between such as 0.25, 0.35, 0.225, 0.335, 0.49; increment ranges there between such as 0.26-0.39; and the like.

In a first embodiment, the present invention relates to compositions and methods for the selective enrichment of low-abundance polynucleotides in a sample. These methods use xeno-nucleic acid (XNA) nucleobase oligomers to selectively block DNA polymerase activity on high abundance wild-type DNA templates, thereby resulting in enrichment of less abundant mutated DNA templates present in a biological sample during a polymerase chain reaction (PCR). The methodology of the present invention can be used to improve DNA sequencing (Sanger sequencing and Pyrosequencing) and also enhance cDNA library preparation for next generation DNA sequencing (NGS).

Utilizing xeno-nucleic acid (XNA) clamping probes in the PCR mediated amplification of DNA templates, only target genetic material that has a variation, e.g. single nucleotide polymorphism (SNP), gene deletion or insertion and/or translocation or truncation is amplified in the oligonucleotide primer directed polymerase chain reaction (qPCR).

The XNA probe clamping sequences are designed to bind specifically by Watson-Crick base pairing to abundant wild-type sequences in the DNA templates derived from the biological sample of interest. The presence of the XNA probes in the PCR primer mix employed for the target amplification reaction causes inhibition of the polymerase mediated amplification of wild-type templates but does not impede the amplification of mutant template sequences.

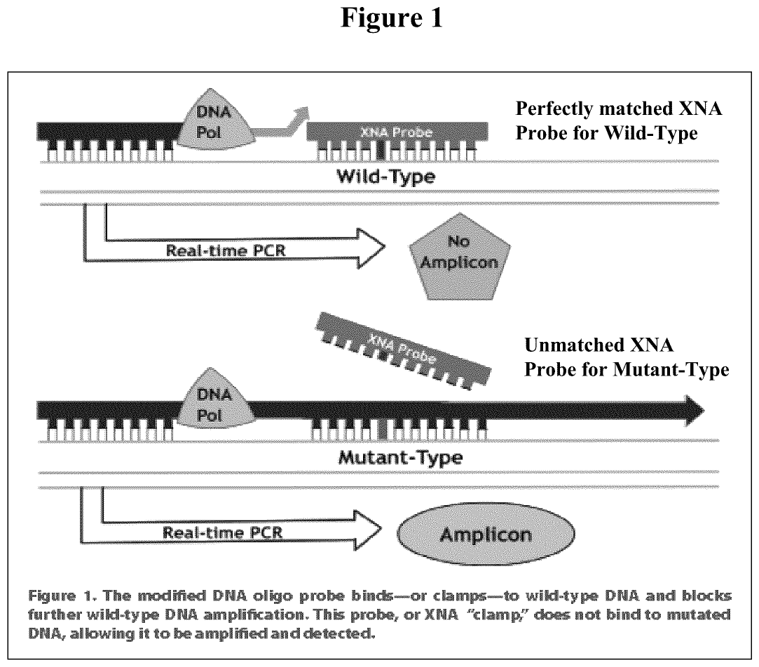

The mechanism of the XNA clamping process is depicted in FIG. 1. As shown in FIG. 1, the modified DNA oligo probe binds or clamps to wild type DNA and blocks further wild type amplification. This probe or XNA “clamp” does not bind to mutated DNA, allowing it to be amplified and detected.

The suppression of wild-type (wt) template amplification and amplification of only mutant templates is achieved because there is a differential melting temperature (Tm) between the XNA clamp bound to mutant templates vs wild type templates:

Tm(XNA mutant template)<<Tm(XNA wt template)

The Tm differential is as much as 15-20° C. for the XNA clamp probes. So that during the PCR process only mutant templates are amplified.

The methods disclosed herein can be used to analyze nucleic acids of samples. The term “sample” as described herein can include bodily fluids (including, but not limited to, blood, urine, feces, serum, lymph, saliva, anal and vaginal secretions, perspiration, peritoneal fluid, pleural fluid, effusions, ascites, and purulent secretions, lavage fluids, drained fluids, brush cytology specimens, biopsy tissue (e.g., tumor samples), explanted medical devices, infected catheters, pus, biofilms and semen) of virtually any organism, with mammalian samples, particularly human samples.

Amplification primers useful in the embodiments disclosed herein are preferably between 10 and 45 nucleotides in length. For example, the primers can be at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or more nucleotides in length. Primers can be provided in any suitable form, included bound to a solid support, liquid, and lyophilized, for example. In some embodiments, the primers and/or probes include oligonucleotides that hybridize to a reference nucleic acid sequence over the entire length of the oligonucleotide sequence. Such sequences can be referred to as “fully complementary” with respect to each other. Where an oligonucleotide is referred to as “substantially complementary” with respect to a nucleic acid sequence herein, the two sequences can be fully complementary, or they may form mismatches upon hybridization, but retain the ability to hybridize under stringent conditions or standard PCR conditions as discussed below. As used herein, the term “standard PCR conditions” include, for example, any of the PCR conditions disclosed herein, or known in the art, as described in, for example, PCR 1: A Practical Approach, M. J. McPherson, P. Quirke, and G. R. Taylor, Ed., (c) 2001, Oxford University Press, Oxford, England, and PCR Protocols: Current Methods and Applications, B. White, Ed., (c) 1993, Humana Press, Totowa, NJ. The amplification primers can be substantially complementary to their annealing region, comprising the specific variant target sequence(s) or the wild type target sequence(s). Accordingly, substantially complementary sequences can refer to sequences ranging in percent identity from 100, 99, 98, 97, 96, 95, 94, 93, 92, 91, 90, 89, 85, 80, 75 or less, or any number in between, compared to the reference sequence. Conditions for enhancing the stringency of amplification reactions and suitable in the embodiments disclosed herein, are well-known to those in the art. A discussion of PCR conditions, and stringency of PCR, can be found, for example in Roux, K. “Optimization and Troubleshooting in PCR,” in Pcr Primer: A Laboratory Manual, Diffenbach, Ed. @ 1995, Cold Spring Harbor Laboratory Press, Cold Spring Harbor, NY; and Datta, et al. (2003) Nucl. Acids Res. 31(19):5590-5597.

Provided herein are methods useful in the detection of sequence variants, i.e., insertions, deletions, nonsense mutations, missense mutations, and the like. In the methods for detecting allelic variants or variant target sequences disclosed herein, the sample, which comprises the nucleic acids to be analyzed, are contacted with an amplification primer pair, i.e., comprising a forward primer and a reverse primer that flank the target sequence or target region containing a sequence of interest {e.g., a wild-type, mutant, or variant allele sequence) to be analyzed. By “flanking” the target sequence, it is understood that the variant or wild-type allelic sequence is located between the forward and reverse primers, and that the binding site of neither the forward nor reverse primer comprises the variant or wild-type allelic sequence to be assessed. For example, in some embodiments, the variant or wild-type allelic sequence to be assessed is removed from or positioned away from the 3′ end of either oligonucleotide by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more, e.g., 100 or more, 200 or more, 300 or more, 400 or more, 500 or more, etc., nucleotides. Amplification primers that flank, but that do not overlap with, the variant target sequence or the wild-type target sequence are thus not “allele-specific” amplification primers, and are capable of amplification of various different alleles or variants of a sequence of interest. Thus, in some embodiments, the amplification primers are configured to amplify various mutant or variant alleles and wild type alleles non-preferentially. As discussed in further detail below, the addition of XNA or PNA to an amplification reaction suppresses the amplification of wild-type target sequences and enables preferential amplification of non-wild-type, e.g., variant, mutant or rare variant alleles. FIG. 1 is a depictions of exemplary method according to the embodiments disclosed herein for the detection of sequence variants. As shown in FIGS. 1, amplification primers (i.e., forward primer 1 and reverse primer 2) flank the wild type and mutant allele sequences of interest, and comprise sequences common to both wild-type and mutant or variant allele sequences. Accordingly, as shown in FIG. 1, in contrast to methods that utilize allele-specific amplification primers to achieve preferential amplification of rare sequences, the present methods advantageously enable the simultaneous amplification of multiple variant sequences, using a single amplification primer pair.

In a second embodiment, the invention relates to compositions and methods for the detection of genetic variations (mutations) in DNA templates derived from biological samples with xeno-nucleic acid clamping probes. The first method employs multi-color fluorescence detection using locus specific fluorescent hybridization probes (Hyb Probes), hydrolysis (TaqMan or ZEN) probes or molecular beacons. The second method employs mutant specific amplicon capture probes immobilized on multiple bar-coded capture beads.

Current XNA clamping qPCR methodologies utilize a single tube-single mutation detection format it is preferable to detect multiple genetic variations in a single tube thus reducing the complexity of the assay and the amount of template DNA required for analysis.

This second embodiment of the invention is directed to the use of locus specific fluorescent probes designed to detect the genetic variant (mutant) amplicons generated during the XNA clamping PCR reaction. This second embodiment discloses locus specific probes that bind to mutant specific amplicons at a region upstream or downstream from the site of the mutation to be detected. Furthermore, the second embodiment discloses the use of multiplexed XNA clamping qPCR reactions that are able to detect multiple mutations (up to a maximum of 6) in one PCR reaction tube using fluorescence detection methodology.

In a third embodiment of the invention, there is provided a method the rapid isolation of genetic material present in circulating cells and also cell-free genetic material from biological fluids and the determination of genetic variations in those cells and biological fluids. Such biological fluids include: blood, serum, plasma, saliva, mucus, urine, sputum, semen or other biological secretions. In this embodiment, the invention also provides the detection of somatic and germ-line mutations in the genetic material derived from these biological fluids utilizing gene amplification and xeno-nucleic acid clamping.

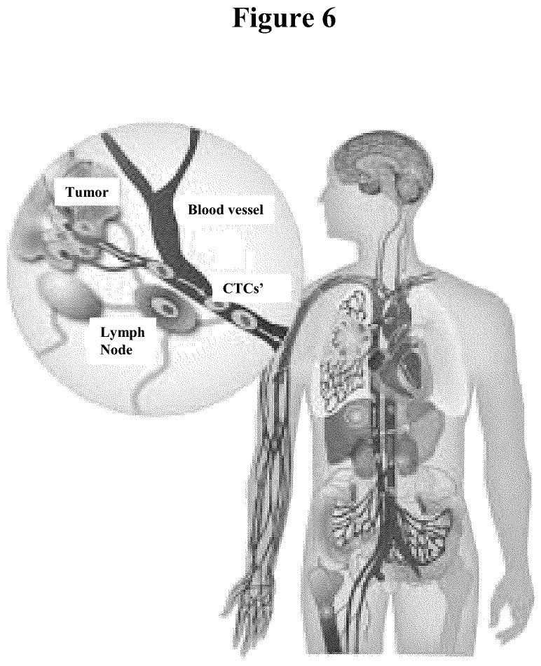

Circulating tumor cells (CTC's) and cell-free DNA (cfDNA) derived from tumor cells are present in the peripheral blood of cancer patients (See FIG. 6). Tumor derived DNA can also be found in the urine and even the saliva of cancer patients.

In general circulating free DNA is smaller in size than DNA derived directly from a surgical biopsy or FFPE sample. This embodiment also describes a novel sample treatment procedure that utilizes a novel lysis reagent called QZol™. QZol™ sample lysis is a direct one tube procedure and an aliquot of the lysate is used directly in molecular genetic and cytogenetic analysis procedures such as PCR, RTPCR, FISH, Next Generation Sequencing (NGS) and branched DNA (bDNA) assays. The QZol™ procedure eliminates the tedious multistep preanalytical processing that is currently used in Molecular Pathology and Cytogenetic analysis.

The lysis reagent is a 50% solution (A) containing chaotropic salts and detergent (nonionic, anionic, cationic or zwitterionic) and a 50% solution (B) containing neutralizing reagents and stabilizers.

This invention also concerns to the specific amplification of genetic variant templates from the isolated genetic material described above. Only target genetic material that has a variation, e.g. single nucleotide polymorphism (SNP), gene deletion or insertion and/or translocation or truncation is amplified in a quantitative primer directed polymerase chain reaction (qPCR). This is achieved utilising xenonucleic acid (XNA) probe clamping sequences that have been designed to bind specifically by Watson-Crick base pairing to wild-type sequences in the sample. The presence of the XNA probes in the qPCR primer mix employed for the target amplification reaction causes inhibition of the polymerase mediated amplification of wild-type templates but does not impede the amplification of mutant template sequences.

The mechanism of the XNA clamping process is depicted in FIG. 1.

The suppression of wild-type (wt) template amplification and amplification of only mutant templates is achieved because there is a differential melting temperature (Tm) between the XNA clamp bound to mutant templates vs wt templates:

Tm(XNA mutant template)<<Tm(XNA wt template)

The Tm differential is as much as 15-20° C. for the XNA clamp probes. So that during the qPCR process only mutant templates are amplified.

The methods disclosed herein can be used in the detection of numerous allelic variants, including nonsense mutations, missense mutations, insertions, deletions, and the like. Owing to the advantageous sensitivity and specificity of detection afforded by the methods disclosed herein, the methods can detect the presence of a rare allelic variant within a sample, amongst a high wild-type background. Accordingly, although the skilled artisan will appreciate that the methods disclosed herein can be used in a variety of settings to detect, e.g., germline mutations, the methods are particularly well-suited for use in the detection of somatic mutations, such as mutations present in tumors. Non-limiting examples of rare, somatic mutations useful in the diagnosis, prognosis, and treatment of various tumors include, for example, mutations in ABL, AKT1, AKT2, ALK, APC, ATM, BRAF, CBL, CDH1, CDK 2A, CEBPA, CRLF2, CSF1R, CTNNB1, EGFR, ERBB2, EZH2, FBXW7, FGFR, FGFR2, FGFR3, FLT3, FOXL2, GATA1, GATA2, GNAQ, GNAS, HNF1A, HRAS, IDH1, IDH3, JAK2, KIT, KRAS, MEK1, MET, MPL, NF2, NOTCH 1, NOTCH2, NPM, NRAS, PC A3, PDGFRA, PIK3CA, PIK3R1, PIK3R5, PTCHI, PTEN, PTPNI 1, RBI, RET, RUNX1, SMAD4, SMARCB, SMO, STK11, TET2, P53, TSHR, VHL, WT1, and others. Exemplary mutant alleles associated with cancer useful in the embodiments disclosed herein include, but are not limited to those described in publications listed on the world wide web site for COSMIC (Catalogue Of Somatic Mutations In Cancer).

### EXAMPLES

### Example 1

The kit described in great detail in this Example is a KRAS mutation detection kit. However, the same type of kit may be assembled to detect mutations in NRAS, EGFR, BRAF, PIK3CA, JAK2, as well as other genes of importance in precision molecular diagnostics.

### QCLAMP™ Technology for Mutation Detection

The QCLAMP™KRAS Mutation Detection Kit is based on xenonucleic acid (XNA) mediated PCR clamping technology. XNA is a synthetic DNA analog in which the phosphodiester backbone has been replaced by a repeat formed by units of (2-aminoethyl)-glycine. XNAs hybridize tightly to complementary DNA target sequences only if the sequence is a complete match. Binding of XNA to its target sequence blocks strand elongation by DNA polymerase. When there is a mutation in the target site, and therefore a mismatch, the XNA:DNA duplex is unstable, allowing strand elongation by DNA polymerase. Addition of an XNA, whose sequence with a complete match to wild-type DNA, to a PCR reaction, blocks amplification of wild-type DNA allowing selective amplification of mutant DNA. XNA oligomers are not recognized by DNA polymerases and cannot be utilized as primers in subsequent real-time PCR reactions.

### DNA Isolation

Human genomic DNA must be extracted from tissue or blood, or fixed paraffin-embedded tissue prior to use. Several methods exist for DNA isolation. For consistency, we recommend using a commercial kit, such as Qiagen DNA extraction kit (QIAamp DNA FFPE Tissue Kit, cat No. 56404, for paraffin embedded specimens; DNeasy Blood & Tissue kit, cat. No. 69504 or 69506, for tissue and blood specimens). Follow the genomic DNA isolation procedure according to manufacturer's protocol. Sufficient amounts of DNA can be isolated from FFPE blocks or fresh frozen sections (approx. 2-10 μg).

This QCLAMP™ assay requires a total of 30-60 ng of DNA per sample (5-10 ng/reaction). After DNA isolation, measure the concentration using spectrophotometric analysis (i.e. Nanodrop or UV spectrophotometer) and dilute to it to 1.25-2.5 ng/μl. Make sure A260/A230 value is greater than 2.0 and A260/A280 value between 1.8 and 2.0.

### Preparation of Reagents

Each kit contains enough material to run 3 sets (10-sample test kit) or 6 sets (30-sample test kit) of Clamping Controls, Positive Controls and Non-Template Controls. Thaw all Primers, XNAs, Positive Control, WT Clamping Control, water and 2× PCR Mastermix provided. Thaw all reaction mixes at room temperature for a minimum of 1 hour. Vortex all components except the PCR Master Mix the reaction mixes for 5 see and perform a quick spin. The PCR Master Mix should be mixed gently by inverting the tube a few times. Do not leave kit components at room temperature for more than 4 hours. After thawing, keep materials on ice at all times. The PCR reactions are set up in a total volume of 20 μl/reaction.

Table 1 shows the component volumes for each 20 ul reaction.

TABLE 1
QCLAMP ™ Sample DNA Preparation Protocol

| Components | Volume/Reaction |
|---|---|
| 2X PCR Master mix | 10 μl |
| Primer Mix | 4 μl |
| XNA | 2 μl |
| DNA sample or Controls | 4 μl |
| Total volume | 20 μl |

For accuracy, 2× PCR Mastermix, primers and XNA should be pre-mixed into assay mixes as described in Table 2 below.

### Preparation of Assay Mixes

IMPORTANT: Assay mixes should be prepared just prior to use. Do not store assay mixes. Prepare and keep assay mixes on ice, until ready for pcr. Label 7 micro centrifuge tubes (not provided) according to each corresponding reaction mix shown in Table 2.

TABLE 2
Preparation of Assay Mixes

| | | | Volume |
|---|---|---|---|
| | | | of XNA |
| | Volume of 2X PCR | Volume of Primer | (†use water |
| | Master Mix | Mix | for ext control) |
| Ext Control Mix | 10 μl × (*n + 1) | 4 μl × (*n + 1) | 2 μl × (*n + 1) |
| G12 Mix | 10 μl × (*n + 1) | 4 μl × (*n + 1) | 2 μl × (*n + 1) |
| G13 Mix | 10 μl × (*n + 1) | 4 μl × (*n + 1) | 2 μl × (*n + 1) |
| A59 Mix | 10 μl × (*n + 1) | 4 μl × (*n + 1) | 2 μl × (*n + 1) |
| Q61 Mix | 10 μl × (*n + 1) | 4 μl × (*n + 1) | 2 μl × (*n + 1) |
| K117 Mix | 10 μl × (*n + 1) | 4 μl × (*n + 1) | 2 μl × (*n + 1) |
| A146 Mix | 10 μl × (*n + 1) | 4 μl × (*n + 1) | 2 μl × (*n + 1) |

*n = number of reactions (DNA samples plus 3 controls). Prepare enough for 1 extra sample (n + 1) to allow for sufficient overage for the PCR set.
†Use 2 ul of water provided in the kit as the Ext Control Mix does not require XNA. For accuracy, do not pipette less than 10 ul of the XNA.

Prepare sufficient working assay mixes for the DNA samples, one KRAS Mixed Positive Control, one Nuclease-Free Water for no template control, and one WT Clamping Control, according to the volumes in Table 2. Include reagents for 1 extra sample to allow sufficient overage for the PCR set up. The master mixes contain all of the components needed for PCR except the sample.

Each sample requires one reaction for each mutation site detected by the kit and an external control. The External Control uses Exon 5 primers to determine if an appropriate level of amplifiable DNA is present in the sample, and ensures that that the supplied primers and polymerase are working properly on the sample. The KRAS Codon-Specific kit requires a total of 7 reactions for each sample.

A set of clamping controls must be run with each of the 7 reaction mixes, every time the assay is run. Clamping Controls use wild-type DNA as the template. Wild-type DNA should have no mutations, therefore the XNA probes will bind strongly, blocking the polymerase from making amplicons. However, the External Control Mix with the Clamping Control should make amplicons efficiently, providing another way to monitor performance of the primers, polymerase, and sample.

A set of positive controls must also be run with each of the 7 reaction mixes, every time the assay is run. The Positive Control contains one mutant template for each reaction mix. Positive controls contain mutations; therefore XNA probes will not bind, allowing amplification of the mutant template. Positive controls must show the appropriate values for the reaction to be valid.

A set of no template control (tube NTC) is run with each of the 7 reaction mixes every time the assay is run. Nuclease-Free Water is used in the place of template. The NTC serves as a negative control and assesses potential contamination during assay set-up.

Further quantities of KRAS Wild-Type Genomic Reference DNA Control, and Positive Control mixes can be purchased as a separate item, if desired.

Suggested Run Layout (96-well plate, tube strips, or tubes)

Gently vortex the assay mixes for 5 see and do a quick spin. Add 16 μl of the appropriate assay mix to the plate or tubes. Add 4 μl of template. Prepare and keep on ice until ready for PCR.

In the case of 96-well plates, the exact plate layout can be set to the user's preference.

However, take care to remember which wells are for which reaction mixes, to ensure that all potential detected mutations and controls are processed properly.

Table 3 is a suggested plate set-up for a single experiment analyzing 3 unknown samples.

TABLE 3
Suggested Plate Layout

| | 1 | 2 | 3 | 4 | 5 | 6 |
|---|---|---|---|---|---|---|
| A | NTC | PC | CC | S1 | S2 | S3 |
| | Ext Ctrl | Ext Ctrl | Ext Ctrl | Ext Ctrl | Ext Ctrl | Ext Ctrl |
| | Mix | Mix | Mix | Mix | Mix | Mix |
| B | NTC | PC | CC | S1 | S2 | S3 |
| | G12 | G12 | G12 | G12 Mix | G12 Mix | G12 Mix |
| | Mix | Mix | Mix | | | |
| C | NTC | PC | CC | S1 | S2 | S3 |
| | G13 | G13 | G13 | G13 Mix | G13 Mix | G13 Mix |
| | Mix | Mix | Mix | | | |
| D | NTC | PC | CC | S1 | S2 | S3 |
| | A59 | A59 | A59 | A59 Mix | A59 Mix | A59 Mix |
| | Mix | Mix | Mix | | | |
| E | NTC | PC | CC | S1 | S2 | S3 |
| | Q61 | Q61 | Q61 | Q61 Mix | Q61 Mix | Q61 Mix |
| | Mix | Mix | Mix | | | |
| F | NTC | PC | CC | S1 | S2 | S3 |
| | K117 | K117 | K117 | K117 Mix | K117 Mix | K117 Mix |
| | Mix | Mix | Mix | | | |
| G | NTC | PC | CC | S1 | S2 | S3 |
| | A146 | A146 | A146 | A146 Mix | A146 Mix | A146 Mix |
| | Mix | Mix | Mix | | | |

PC: Positive Control,
NTC: No Template Control (water),
CC: Clamping Control (Wild-type DNA),
S1-3: Samples 1-3.
NOTE:
For setup on the Rotor-Gene Q Platforms, the layout must be changed such that the first well contains Positive Control.

When all reagents have been loaded, tightly close the PCR tubes or seal the 96-well plate to prevent evaporation. Spin at 2000 rpm for 1 minute to collect all the reagents. Place in the real-time PCR instrument immediately or store on ice until the instrument is ready.

### Instrument Set-Up

### Roche LightCycler 96 or RocheLightCycler 480

1. Select New empty experiment>create
2. In the Run Editor>Measurement, choose SYBR Green 1 (470/514) channel on (LC96), SYBR Green 1/HRM Dye on (LC480)
3. Set up run profile using parameters in Table 7. Ramp rates for the LC 96 and LC480 should match settings below.
4. During the analysis set threshold to Auto.

TABLE 4
Roche Light Cycler, LC96 and LC480 Parameters

| | Temper- | Time | | | | |
|---|---|---|---|---|---|---|
| | ature | (Sec- | | Ramp | | Acquisition |
| Step | (° C.) | onds) | Cycles | Rate | Mode | Mode |
| PreIncubation | 95 | 300 | 1 | 4.4 | | None |
| Denaturation | 95 | 20 | | 2.2 | Standard | None |
| XNA | 70 | 40 | X40 | 2.2 | | None |
| Annealing | | | | | | |
| Primer | 64 | 30 | | 2.2 | | None |
| Annealing | | | | | | |
| Extension | 72 | 30 | | 1.0 | | Single |
| Melting | 95 | 10 | 1 | 4.4 | | None |
| | 65 | 60 | | 2.2 | | None |
| | 97 | 1 | | 0.20 | | Continuous |
| | | | | | | (5 readings/ |
| | | | | | | ° C.) |
| Cooling | 37 | 30 | 1 | 2.2 | | None |

* An HRM curve or melt analysis should be run at the end of the PCR reaction. This helps to verify the PCR amplification results and with troubleshooting.

### Applied Biosystems Platforms

1. Select File>New Experiment
2. Enter an experiment name and select 7500 (96 wells) or as appropriate
3. Select Quantitation-Standard Curve
4. Select SYBR Green Reagents
5. Select Standard Ramp Rate if available
6. Click on Plate Setup in the left navigation panel
7. Select the Assign Targets and Samples tab and assign samples to the wells
8. Select NONE for the Passive Reference Dye
9. Click on Run Method on the left panel, set reaction volume to 20 ul
10. Setup the cycling parameters as shown in the table below
11. Add Melt Curve at the end of the Cycling Stage. Use continuous and leave default setting for data collection
12. During the analysis set threshold to 0.5 (ABI 7900) and 5000 (ABI 7500).

TABLE 5
Applied Biosystems Platforms Cycling Parameters

| | | Time | | Data |
|---|---|---|---|---|
| Step | Temperature (° C.) | (Seconds) | Cycles | Collection |
| PreIncubation | 95 | 300 | 1 | OFF |
| Denaturation | 95 | 20 | X40 | OFF |
| XNA Annealing | 70 | 40 | | OFF |
| Primer Annealing | 66 | 30 | | OFF |
| Extension | 72 | 30 | | ON |
| Melt Curve | Default | | | Continuous |

### Rotor-Gene Q Platforms

In the instrument software version 2.1 and above
1. Select File>New, Select Three Step with Melt and click New
2. Select 72-Well Rotor, check the Locking Ring Attached box, click Next
3. Set Reaction volume to 20 ul, click next
4. Set Temperature profile as shown in Table 6.
5. Channel Setup: Select Green Source 470 nm, Detector 510 nm, Gain 7
a. Click Gain Optimization
b. Set Temperature to 70 C
c. Perform Optimization before 1st acquisition
d. Click optimize acquiring
e. In the pop-up box enter
i. Target Sample Range 5 FL up to 10 FL
ii. Acceptable Gain Range-10 to 10
f. Click OK, Click Close, Click Next
6. Start-run
7. During the analysis set threshold to Auto.

TABLE 6
Rotor-Gene Q Platforms Cycling Parameters

| | | | | | |
|---|---|---|---|---|---|
| Hold | | 95° C. | 5 minutes | X1 | Not Acquiring |
| Cycling | Timed Step | 95° C. | 20 seconds | X40 | Not Acquiring |
| | Timed Step | 70° C. | 40 seconds | | Not Acquiring |
| | Timed Step | 64° C. | 30 seconds | | Not Acquiring |
| | Timed Step | 72° C. | 40 seconds | | Acquiring to |
| | | | | | Cycling A |
| | | | | | on Green |

| | | |
|---|---|---|
| Melt | Ramp from 65 to 95, rising | Acquire to melt |
| | by 1 degree each step | A on green |
| | Wait for 90 sec of pre-melt | |
| | conditioning on first step | |
| | Wait for 5 seconds for | |
| | each step afterwards | |
| | Gain Optimization | |
| | Check optimize gain before | |
| | melt on all tubes | |
| | The gain giving the highest | |
| | fluorescence less than 95 | |
| | will be selected. | |

### Assessment of Real-Time PCR Results

For the analysis use Absolute Quantitation, automatic baseline. The threshold to be used with each instrument is listed above. Check threshold to ensure that the Threshold is within the exponential growth phase of the amplification plot. If not, the threshold may be adjusted depending on the run.

The real-time PCR instrument generates a Cq value. Cq is the cycle threshold, the cycle number at which a signal is detected above background fluorescence. The lower the cycle number at which signal rises above background, the stronger the PCR reaction it represents

### No Template Controls

Verify that there is no amplification in no-template controls for each of the reaction mixes. Cq should be undetermined. For some mixes a Cq of 36 or higher may be observed in the NTC. In such cases, check the melting curves obtained. If the melting curve indicates the presence of primer dimers, the reaction may be acceptable. SYBR green binds to primer dimers, resulting in a peak with a lower melting temperature, than the desired amplicon. In many cases formation of primer dimers can be avoided by setting up the PCR reactions on ice, until ready to load into the PCR instrument.

### Analysis of Clamping and Positive Controls

The Cq values of the Positive Control (mixed mutant templates) should amplify in the presence of XNAs and yield Cq values given in Table 7.

TABLE 7
Acceptable Cq Ranges for Positive Controls

| | Positive Control |
|---|---|
| | Acceptable Cq Range |
| Ext Control | 20 ≤ Cq ≤ 26 |
| G12 Mix | ≤32 |
| G13 Mix | ≤32 |
| A59 Mix | ≤32 |
| Q61 Mix | ≤30 |
| K117 Mix | ≤34 |
| A146 Mix | ≤30 |

*The Cq value of the Clamping Control (WT DNA) with the Ext Control Mix should be within 20 and 26.
*In addition, the Cq of the Clamping Control with each of the mutation reaction mixes should be at least 3 Cq greater than the Cq of Positive Control with the same reaction mix. If these criteria are not met, the reaction has failed and the results are not valid.
PASS: Cq of Clamping Control with mutation reaction mix - Cq of Positive Control with same mutation reaction mix ≥ 3
FAIL: Cq of Clamping Control with mutation reaction mix - Cq of Positive Control with same mutation reaction mix ≤ 3

### Judging Validity of Sample Data Based on External Control Mix Results

The Cq value of the Ext Control Mix can serve as an indication of the purity and the concentration of DNA. Thus, the validity of the test can be decided by the Cq value of the Ext Control Mix. Cq values of any sample with Ext Control Mix should be in the range of 20-27. If the Cq values fall outside the range given in Table 8, the test results should be considered invalid. The experiment should be repeated.

TABLE 8
Acceptable Cq Ranges for Samples with External Control Mix

| | Cq Value of Ext | |
|---|---|---|
| Validity | Control Mix | Descriptions and Recommendations |
| Optimal | 20 < Cq < 27 | The amplification and amount of DNA sample were optimal. |
| Invalid | Cq ≤ 20 | Possibility of a false positive is high. Repeat the PCR reaction with |
| | | less DNA. |
| Invalid | Cq ≥ 27 | Not enough DNA or DNA not pure. The amplification is not |
| | | optimal. Check DNA amount and purity. Repeat the experiment with |

### Scoring Mutational Status

IMPORTANT: Refer to the Macro Sheet for QCLAMP™ Cq Mutation Analysis for scoring mutational status. Macro may be requested by contacting information@diacarta.com
If a Cq value is undetermined, assign a Cq of 40 and proceed to analysis.
The table below should be used to determine mutational status

TABLE 9
Scoring Mutational Status

| Mutation | | G12 | G13 | A59 | Q61 | K117 | A146 |
|---|---|---|---|---|---|---|---|
| Strong | Cq | ≤32 | ≤32 | ≤32 | ≤30 | ≤33 | ≤30 |
| Positive: | | | | | | | |
| Mutation | | | | | | | |
| Content > | | | | | | | |
| 5% | | | | | | | |
| Weak | Cq | 32-35* | 32-35* | 30-35* | 30-35* | 33-35* | 30-35* |
| Positive: | ΔCq | ≤10 | ≤9 | ≤8 | ≤8 | ≤10 | ≤8 |
| Mutation | | | | | | | |
| Content | | | | | | | |
| 1-5% | | | | | | | |
| Negative | Cq | ≥35 | ≥35 | ≤30 | ≥35 | ≥35 | ≥35 |

*If reaction has been set-up with 5 ng of DNA, it is recommended that the experiment be repeated with 10 ng of template DNA to confirm the results.
*Refer to Table 9 for interpretation of A59/Q61 Mutational Status If the Cq value suggests mutation content between 1%-5%, a further calculation of ΔCq should be performed to determine mutational status.
ΔCq = [Cq value of sample with mutant reaction mix] − [Cq value of sample with Ext Control Mix]
For ex: ΔCq = [Cq of sample with G12 mutant reaction mix] − [Cq of sample with Ext Control Mix]

Refer to the table above to confirm mutational status of weak positives.

### Differentiating A59/Q61 Mutational Status

The Q61 reaction mix detects both A59 and Q61 mutations, whereas the A59 reaction mix detects only A59 mutations. Therefore, in order to differentiate between A59 and Q61 Mutations a combination of results from the 2 mixes should be used, as described in Table 10 below.

TABLE 10
Interpretation of A59/Q61 Mutational Status

| Reaction Mix | Result Based on Table 12 | Mutational Status |
|---|---|---|
| A59 Reaction Mix | Positive | A59 Mutation |
| Q61 Reaction Mix | Positive | |
| A59 Reaction Mix | Negative | Q61 Mutation |
| Q61 Reaction Mix | Positive | |
| A59 Reaction Mix | Negative | Q61 Mutation |
| Q61 Reaction Mix | Positive | |

### HRM Curves as a Tool to Confirm Analyses

In High Resolution Melting Analysis (HRM), the region of interest amplified by PCR is gradually melted. SYBR green is a dsDNA binding dye that is released as the dsDNA amplicon is melted. Emitted fluorescence is measured to generate a characteristic curve. The Tm (Melting Temperature) is characteristic of the GC content, length and sequence of a DNA product and is a useful tool in product identification. The resulting melt profile reflects the mix of amplicons present.

Wild-type DNA (clamping control) is provided. Some amplification may occur in these reactions. Melt profiles of unknown samples should be compared to wild-type and positive controls. Enrichment of one or more peaks, resulting in a melt profile distinct from wild-type DNA profile, can serve as an indication of specific amplification of a mutation target. If the melt profile of an unknown sample is similar to wild-type DNA, and has been scored as a mutation due to Cq, the analysis should be repeated. The resulting PCR product can be sent for Sanger sequencing for further clarification.

HRM curves obtained from unknown samples can be compared to HRM curves obtained from positive controls. Amplicons of similar length and sequence will exhibit the same melt profile.

### Example 2

PCR based enrichment of mutant DNA template sequences from template DNA derived from a lung cancer tumor biopsy sample is shown below using a xeno-nucleic acid clamping probe specific for KRAS Exon 2 codon 12. Only codon 12 mutant sequences are amplified as shown by the melting profile of the PCR amplicons generated before enrichment and after XNA clamped PCR enrichment:

The PCR product from the XNA clamped mutant enriched PCR reaction can be isolated and used directly in a Sanger sequencing or Pyrosequencing reaction or else it can be processed for next generation sequencing (NGS) by ligation of adapters and after removal of excess adapters can be used directly for NGS without the need for another PCR amplification step.

### Example 3

### Multiplex Detection of KRAS Mutations.

In this example of this invention, locus specific hydrolysis probes are designed to detect mutant amplicons in the KRAS proto-oncogene. Locus specific probes are designed for the following mutant amplicons in KRAS:
Probe 1 KRAS Exon 2 codon 12,
Probe 2 KRAS Exon 2 codon 13,
Probe 3 KRAS Exon 3 codon 59
Probe 4 KRAS Exon 3 codon 61,
Probe 5 KRAS Exon 4 codon 117,
Probe 6 KRAS Exon 4 codon 146
and a control probe for a coding sequence in KRAS that has no mutations-Probe 7 KRAS Control probe

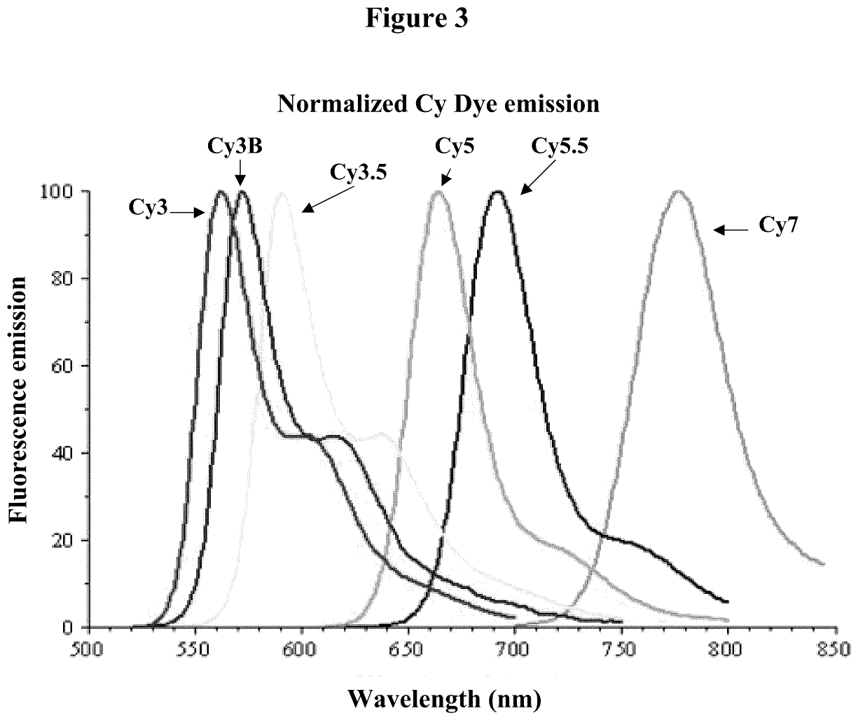

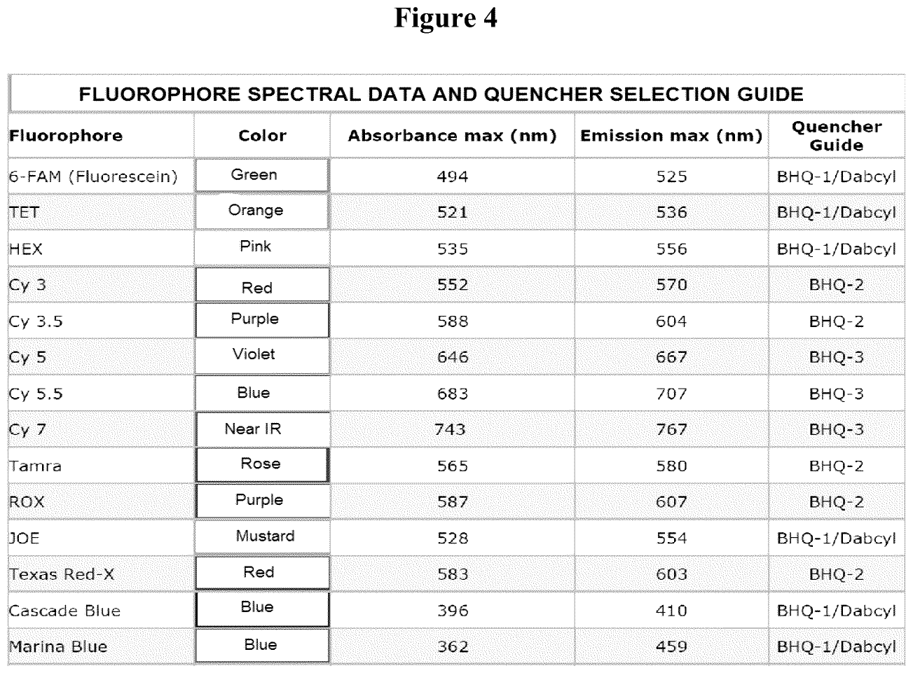

Each locus specific hydrolysis probe has a different fluorophore (and quencher) selected from the available fluorophores for multiplex applications (see FIGS. 3 and 4).

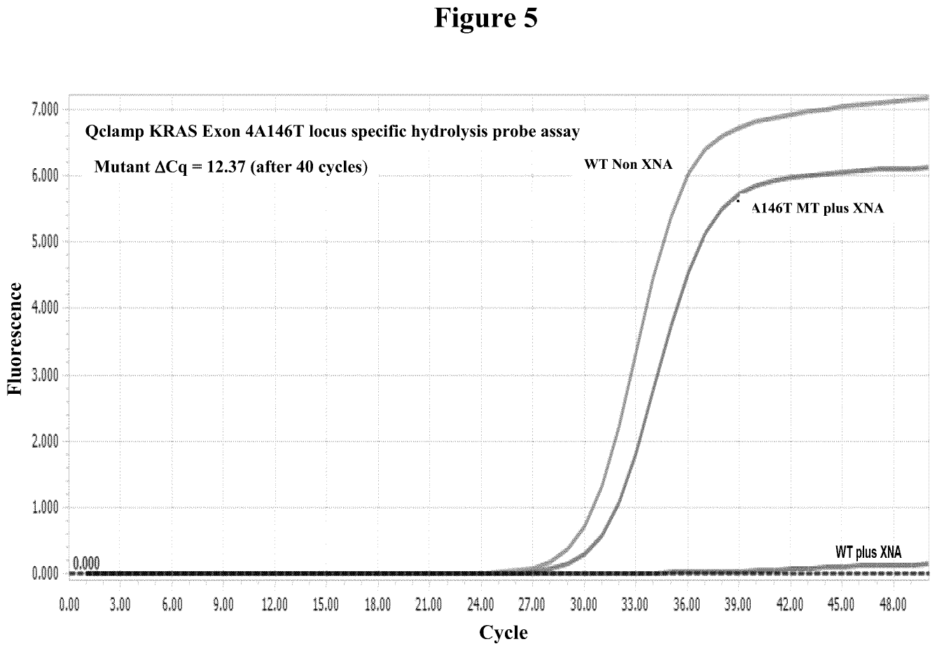

For the KRAS multiplex assay, KRAS c12, c59, c117 and c146 and KRAS control are detected in a one tube and KRAS c13 and c61 and KRAS control in a separate tube. So that all mutations in the KRAS proto-oncogene can be detected using only 2 PCR reaction tubes. FIG. 5 is an Example of the Exon 4 locus specific probes assay.

### Example 4

This example of the invention describes the use of mutation specific capture probes covalently attached to optically bar-coded beads via an amino-linker spacer. Mutant specific probes and control probes for the detection of mutations in KRAS Exon 2 codons 12 and 13 are shown below:

```
  1. G12A
SEQ ID NO: 1
AGCTGCTGGCGTA
  2. G12R
SEQ ID NO: 2
AGCTCGTGGCGTA
  3. G12D
SEQ ID NO: 3
AGCTGATGGCGTA
  4. G12C
SEQ ID NO: 4
AGCTTGTGGCGTA
  5. G12I
SEQ ID NO: 5
GAGCTATTGGCGT
  6. G12L
SEQ ID NO: 6
GAGCTCTTGGCGT
  7. G12S
SEQ ID NO: 7
AGCTAGTGGCGTA
  8. G12V
SEQ ID NO: 8
AGCTGTTGGCGTA
  9. G13C
SEQ ID NO: 9
TGGTTGCGTAGGC
10. G13D
SEQ ID NO: 10
TGGTGACGTAGGC
11. G13A
SEQ ID NO: 11
TGGTGCCGTAGGC
12. G13V
SEQ ID NO: 12
TGGTGTCGTAGGC
13. G13S
SEQ ID NO: 13
TGGTAGCGTAGGC
14. G13R
SEQ ID NO: 14
TGGTCGCGTAGGC
```

15. The control Capture Probes are:

16. (HLA-)DRA Match
SEQ ID NO: 15
GGAGACGGTCTGG
17. (HLA-)DRA Mismatch
SEQ ID NO: 16
GGAGACGCTCTGG
18. KRAS Wild type:
SEQ ID NO: 17
CTGGTGGCGTAGG
19. KRAS PCR control
SEQ ID NO: 18
AAGGCCTGCTGAA

All probes contain a 5′-amino-linker for bar-coded bead conjugation. After, performing XNA clamping PCR reaction is done to eliminate wild-type KRAS using the following primers:

KRAS Exon 2 Forward: SEQ ID NO: 19 5′-GTACTGGTGGAGTATTTGATAGTG-3′ KRAS Exon 2 Reverse: SEQ ID NO: 20 5′-ATCGTCAAGGCACTCTTGCCTAC-3′ and XNA Clamp Probe Blocker specific for KRAS Exon 2 12/13 optically bar-coded mutation specific capture beads are added and incubated for hybridization capture. After washing detection is performed with Streptavidin Phycoerythrin (SAPE) and measured on DigiPlex analyzer.

### Example 5

### QCLAMP™ Sample DNA Preparation Protocol

Genomic DNA should be obtained either from whole blood, cells, purified peripheral blood lymphocytes of whole blood, polynuclear cells, or granulocytes, tissue biopsies or FFPE sections. For comparable results it is recommended that the same cellular fraction and DNA extraction method are used. DNA extraction can be performed using a homebrew method or a commercially available kit.

Carefully transfer FFPE section(s) or equivalent amount of fresh tissue, cells (100 to 100,000 cells) or 200 μl whole blood to a clean 1.7 ml polypropylene micro-centrifuge tube and add the required volume of lysis solution. For FFPE sections add 50 μL of lysis Solution. For liquid or moist cells or tissues add 2× volume of the sample volume.

For FFPE samples warm each sample in heating block at 95° C. until paraffin melts and then vortex each warm sample for 10 seconds. Return the sealed sample preparation tubes to the heating block and heat at 95° C. for 20 minutes make sure to carefully remove the tubes every 5 min and vortex each tube for 10 s and return to heating block.

Remove sample preparation tube from heating block and immediately add an equivalent volume of lysis solution as the volume added of lysis solution from step 1 above. For example, if 50 μL of lysis solution was added, add 50 L of lysis solution.

Vortex each sample for 10 seconds. Spin down the sample preparation tubes in a microcentrifuge and allow to cool. Use the resultant lysis solution lysate supernatant directly in the PCR reaction.

The extracted DNA needs to be diluted to a concentration of 5 ng/μl in 1× TE buffer at pH 8.0 and then stored at +4 to +8° C. for 1 week or at −20° C. if longer term storage is required. The QCLAMP™ qPCR reaction is optimized for DNA samples containing 5-20 ng of purified genomic DNA.

The sequences in the Table below show exemplary primers and xeno and peptide nucleic acids.

| Sequence Name | | |
|---|---|---|
| 1047SSF001NEW | SEQ ID NO: 21 | CGAAAGACCCTAGCCTTAGATAAAACT |
| 1047SSR001NEW | SEQ ID NO: 22 | ATTGTGTGGAAGATCCAATCCATTT |
| 146R002f | SEQ ID NO: 23 | ACGTTGGATGTGTACCATACCTGTCTGGTCTT |
| 21FW1S | SEQ ID NO: 24 | GTTTTCCCAGTCACGACACGTTGGATGCAGCCAGG |
| | | AACGTACTGGTGA |
| BIOBRAFCONTRLFP | SEQ ID NO: 25 | /5Biosg/CTCCAGATCTCAGTAAGGTACGG |
| BIOKRASCONTRLFP | SEQ ID NO: 26 | /5Biosg/TGAGGGAGATCCGACAATACAG |
| BRAFAZFPNEW02 | SEQ ID NO: 27 | ACAGTAAAAATAGGTGATTTTGGTCTAGCTA |
| BRAFAZFPNew02s | SEQ ID NO: 28 | GTTTTCCCAGTCACGACACGTTGGATGACAGTAAA |
| | | AATAGGTGATTTTGGTCTAGCTA |
| BRAFAZRP001 | SEQ ID NO: 29 | CATCCACAAAATGGATCCAGACAA |
| BRAFAZRP001s | SEQ ID NO: 30 | CAGGAAACAGCTATGACACGTTGGATGCATCCACA |
| | | AAATGGATCCAGACAA |
| BRAFCONTRLFP | SEQ ID NO: 31 | CTCCAGATCTCAGTAAGGTACGG |
| BRAFCONTRLRP | SEQ ID NO: 32 | GGGAAAGAGTGGTCTCTCATC |
| C790F002f | SEQ ID NO: 33 | ACGTTGGATGTCCACCGTGCAGCTCATC |
| C790F002fS | SEQ ID NO: 34 | GTTTTCCCAGTCACGACACGTTGGATGTCCACCGT |
| | | GCAGCT |
| C790R001Bf | SEQ ID NO: 35 | ACGTTGGATGGTCTTTGTGTTCCCGGACAT |
| C790R001BfS | SEQ ID NO: 36 | CAGGAAACAGCTATGACACGTTGGATGGTCTTTGT |
| | | GTTCCC |
| Ex19NewFS | SEQ ID NO: 37 | GTTTTCCCAGTCACGACACGTTGGATGCTCTCTGT |
| | | CATAGGGACTCTGGATCC |
| Ex19NewFwd | SEQ ID NO: 38 | CTCTCTGTCATAGGGACTCTGGATCC |
| Ex19NewRev | SEQ ID NO: 39 | AGCAAAGCAGAAACTCACATCGAG |
| Ex19NewRS | SEQ ID NO: 40 | CAGGAAACAGCTATGACACGTTGGATGAGCAAAGC |
| | | AGAAACTCACATCGAG |
| Exon18NewFS | SEQ ID NO: 41 | GTTTTCCCAGTCACGACACGTTGGATGGCTCCCAA |
| | | CCAAGCTCTCTTGA |
| Exon18NewFwd | SEQ ID NO: 42 | GCTCCCAACCAAGCTCTCTTGA |
| Exon18NewRev | SEQ ID NO: 43 | CTGTGCCAGGGACCTTACCTTATAC |
| Exon18NewRS | SEQ ID NO: 44 | CAGGAAACAGCTATGACACGTTGGATGCTGTGCCA |
| | | GGGACCTTACCTTATAC |
| Exon2FowardNew | SEQ ID NO: 45 | TTTGCCAAGGCACGAGTAACAAG |
| Exon2ReverseNew | SEQ ID NO: 46 | CCCAAGGACCACCTCACAGTTAT |
| JAK2XN9F001 | SEQ ID NO: 47 | TTAACTGCAGATGCACATCATTACCT |
| KRAS117F002 | SEQ ID NO: 48 | GGACTCTGAAGATGTACCTATGG |
| KRAS117F002s | SEQ ID NO: 49 | GTTTTCCCAGTCACGACACGTTGGATGGGACTCTG |
| | | AAGATGTACCTATGG |
| KRAS117R002 | SEQ ID NO: 50 | GCTAAGTCCTGAGCCTGTTT |
| KRAS117R002s | SEQ ID NO: 51 | CAGGAAACAGCTATGACACGTTGGATGGCTAAGTC |
| | | CTGAGCCTGTTT |
| KRAS146F003 | SEQ ID NO: 52 | ACACAAAACAGGCTCAGGAC |
| KRAS146F003s | SEQ ID NO: 53 | GTTTTCCCAGTCACGACACGTTGGATGACACAAAA |
| | | CAGGCTCAGGAC |
| KRAS146R002 | SEQ ID NO: 54 | CAGTGTTACTTACCTGTCTTGTCTT |
| KRAS146R002s | SEQ ID NO: 55 | CAGGAAACAGCTATGACACGTTGGATGCAGTGTTA |
| | | CTTACCTGTCTTGTCTT |
| KRASBIOFP002 | SEQ ID NO: 56 | AAGGCCTGCTGAAAATGACTG |
| KRASBioFP002s | SEQ ID NO: 57 | GTTTTCCCAGTCACGACACGTTGGATGAAGGCCTG |
| | | CTGAAAATGACTG |
| KRASC12RP002s | SEQ ID NO: 58 | CAGGAAACAGCTATGACACGTTGGATGTCAAGGCA |
| | | CTCTTGCCTACGC |
| KRASc13F001 | SEQ ID NO: 59 | ACTTGTGGTAGTTGGAGCTGGT |
| KRASC13F001s | SEQ ID NO: 60 | GTTTTCCCAGTCACGACACGTTGGATGACTTGTGG |
| | | TAGTTGGAGCTGGT |
| KRASC13NEWR001 | SEQ ID NO: 61 | TCATGAAAATGGTCAGAGAAACCTT |
| KRASC13NewR001s | SEQ ID NO: 62 | CAGGAAACAGCTATGACACGTTGGATGACTTGTGG |
| | | TAGTTGGAGCTGGT |
| KRASC59R001 | SEQ ID NO: 63 | ATTGCACTGTACTCCTCTTGACC |
| KRASC59R001s | SEQ ID NO: 64 | CAGGAAACAGCTATGACACGTTGGATGATTGCACT |
| | | GTACTCCTCTTGACC |
| KRASc61F001 | SEQ ID NO: 65 | CTCTTGGATATTCTCGACACAGCAGGT |
| KRASC61F001s | SEQ ID NO: 66 | GTTTTCCCAGTCACGACACGTTGGATGCTCTTGGATA |
| | | TTCTCGACACAGCAGGT |
| KRASc61F003 | SEQ ID NO: 67 | CCAGACTGTGTTTCTCCCTT |
| KRASC61F003s | SEQ ID NO: 68 | GTTTTCCCAGTCACGACACGTTGGATGCCAGACTGTG |
| | | TTTCTCCCTT |
| KRASCONTRLFP | SEQ ID NO: 69 | TGAGGGAGATCCGACAATACAG |
| KRASCONTRLRP | SEQ ID NO: 70 | TCTGCCAAAATTAATGTGCTGAACT |
| L858RBR001 | SEQ ID NO: 71 | TTCTCTTCCGCACCCAGC |
| L858RBR001S | SEQ ID NO: 72 | CAGGAAACAGCTATGACACGTTGGATGTTCTCTTCCG |
| | | CACCCAGC |
| L858RNewFS | SEQ ID NO: 73 | GTTTTCCCAGTCACGACACGTTGGATGTGAAAACAC |
| | | CGCAGCATGTCAAGA |
| L858RNewFwd | SEQ ID NO: 74 | TGAAAACACCGCAGCATGTCAAGA |
| L858RNewRev | SEQ ID NO: 75 | CCTTACTTTGCCTCCTTCTGCATG |
| L858RNewRS | SEQ ID NO: 76 | CAGGAAACAGCTATGACACGTTGGATGCCTTACTTT |
| | | GCCTCCTTCTGCATG |
| NC12FP004 | SEQ ID NO: 77 | TGGTGGGATCATATTCATCTACAAAG |
| NC12FP004_G13_Rev | SEQ ID NO: 78 | TGGTGGGATCATATTCATCTACAAAG |
| NC12FP004s | SEQ ID NO: 79 | CAGGAAACAGCTATGACACGTTGGATGTGGTGGGAT |
| | | CATATTCATCTACAAAG |
| NRAS117F001 | SEQ ID NO: 80 | AGTAAAAGACTCGGATGATGTACCTAT |
| NRAS117F002f | SEQ ID NO: 81 | ACGTTGGATGACCTATGGTGCTAGTGGGAAAC |
| NRAS117F003 | SEQ ID NO: 82 | ACGTTGGATGTCCCGTTTTTAGGGAGCAGA |
| NRAS117F004 | SEQ ID NO: 83 | CCCGTTTTTAGGGAGCAGAT |
| NRAS117R002 | SEQ ID NO: 84 | CAGTTCGTGGGCTTGTTTTG |
| NRAS117R004 | SEQ ID NO: 85 | CTTGCACAAATGCTGAAAGC |
| NRASc12F001 | SEQ ID NO: 86 | AAACTGGTGGTGGTTGGAGCA |
| NRASC12F001s | SEQ ID NO: 87 | GTTTTCCCAGTCACGACACGTTGGATGAAACTGGTG |
| | | GTGGTTGGAGCA |
| NRASC13F001 | SEQ ID NO: 88 | GGTGGTGGTTGGAGCAGGT |
| NRASC13F001s | SEQ ID NO: 89 | GTTTTCCCAGTCACGACACGTTGGATGGGTGGTGGTT |
| | | GGAGCAGGT |
| NRASC59F001 | SEQ ID NO: 90 | ACACCCCCAGGATTCTTACAGA |
| NRASC59F001s | SEQ ID NO: 91 | GTTTTCCCAGTCACGACACGTTGGATGACACCCCCAG |
| | | GATTCTTACAGA |
| NRASC59R001 | SEQ ID NO: 92 | ATGGCACTGTACTCTTCTTGTCC |
| NRASC59R001s | SEQ ID NO: 93 | CAGGAAACAGCTATGACACGTTGGATGATGGCACTG |
| | | TACTCTTCTTGTCC |
| NRASc61F001 | SEQ ID NO: 94 | GTTGGACATACTGGATACAGCTGGA |
| NRASC61F001s | SEQ ID NO: 95 | GTTTTCCCAGTCACGACACGTTGGATGGTTGGACATA |
| | | CTGGATACAGCTGGA |
| NRASXN3REVSet4 | SEQ ID NO: 96 | CCGCAAATGACTTGCTATTA |
| NRASXN3RevSet4s | SEQ ID NO: 97 | CAGGAAACAGCTATGACACGTTGGATGCCGCAAATG |
| | | ACTTGCTATTA |
| NRASXN5FwSet1 | SEQ ID NO: 98 | ACACACTGGTAAGAGAAATAC |
| NRASXN5REVSet1 | SEQ ID NO: 99 | CTGAGTCCCATCATCACT |
| BR001 | SEQ ID NO: 100 | ATCGAGATTTCACTGTAGCTAGAC |
| DPCA001 | SEQ ID NO: 101 | ACTTCAGGCAGCGTCTTCA |
| DPCA002 | SEQ ID NO: 102 | TGTTCAGAGCACACTTCAG |
| DPCA003 | SEQ ID NO: 103 | CTGGTGGTTGAATTTGCTG |
| DPCA004 | SEQ ID NO: 104 | CATGAGCTCCAGCAGGATGAAC |
| DPCA005 | SEQ ID NO: 105 | CCGAAGTCTCCAATCTTGG |
| DPCA006 | SEQ ID NO: 106 | TAGATGTCTCGGGCCATCC |
| DPCBRC001 | SEQ ID NO: 107 | GGGACACTCTAAGAT |
| DPCBRC002 | SEQ ID NO: 108 | TTCTGTCCTGGGATTCTC |
| DPCBRC003 | SEQ ID NO: 109 | AGATTTTCCACTTGCTGT |
| DPCBRCA001-2 | SEQ ID NO: 110 | CCAGATGGGACACTCTAAGATTTTC |
| DPCBRCA002-2 | SEQ ID NO: 111 | CCTTTCTGTCCTGGGATTCTCTT |
| DPCBRCA003-2 | SEQ ID NO: 112 | GACAGATTTTCCACTTGCTGTGCTAA |
| DPCBRCA004 | SEQ ID NO: 113 | CATAAAGGACACTGTGAAGGCC |
| DPCBRCA004B | SEQ ID NO: 114 | D-LYS-O-GGCCTTCACAGTGTCCTTTATG |
| DPCCKT002 | SEQ ID NO: 115 | D-LYS-O-CATTCTTGATGTCTCTGGCTAG |
| DPCE001 | SEQ ID NO: 116 | GAGCCCAGCACTTT |
| DPCE001B | SEQ ID NO: 117 | D-LYS-O-CGGAGCCCAGCACTTTGAT |
| DPCE001B1 | SEQ ID NO: 118 | D-LYS-O-CGGAGCCCAGCACTTTGAT |
| DPCE002 | SEQ ID NO: 119 | NH(2)-AGATGTTGCTTCTCTTAA-CONH(2) |
| DPCE002B | SEQ ID NO: 120 | D-LYS-O-AGATGTTGCTTCTCTTAA |
| DPCE002C | SEQ ID NO: 121 | D-LYS-O-CGGAGATGTTGCTTCTCTTAATTCC |
| DPCE004 | SEQ ID NO: 122 | CAGTTTGGCCAGCCCA |
| DPCE004B | SEQ ID NO: 123 | CAGTTTGGCCAGCCCA-O-D-LYS |
| DPCE004C | SEQ ID NO: 124 | D-LYS-O-TTTGGCCAGCCCAAAATCTGT |
| DPCE004D | SEQ ID NO: 125 | D-LYS-O-GGCCAGCCCAAAATCTGT |
| DPCE005 | SEQ ID NO: 126 | ACCCAGCAGTTTGGC |
| DPCE005B | SEQ ID NO: 127 | D-LYS-O-ACCCAGCAGTTTGGC |
| DPCE006 | SEQ ID NO: 128 | GCTGCGTGATGAG |
| DPCE007 | SEQ ID NO: 129 | GCTGCGTGATGA |
| DPCE008 | SEQ ID NO: 130 | AGCTCATCACGCAGCTCATG |
| DPCE008B | SEQ ID NO: 131 | D-LYS-O-CAGCTCATCACGCAGCTCATGC |
| DPCE008C | SEQ ID NO: 132 | D-LYS-O-TCATCACGCAGCTCATGCCCTT |
| DPCE008D | SEQ ID NO: 133 | D-LYS-O-CTCATCACGCAGCTCATG |
| DPCE008E | SEQ ID NO: 134 | D-LYS-O-TGAGCTGCGTGATG |
| DPCE009B | SEQ ID NO: 135 | D-LYS-O-TCCACGCTGGCCATCACGTA |
| DPCE009B-1 | SEQ ID NO: 136 | TCCACGCTGGCCATCACGTA-O-D-LYS |
| DPCE010B | SEQ ID NO: 137 | TGGGGGTTGTCCAC-O-D-LYS |
| DPCE011 | SEQ ID NO: 138 | GCACACGTGGGGGTT-O-D-LYS |
| DPCE012 | SEQ ID NO: 139 | D-LYS-O-ACAACCCCCACGTGTGC |
| DPCH001 | SEQ ID NO: 140 | CTGAGCCAGGAGAAAC |
| DPCH002 | SEQ ID NO: 141 | GTAAACTGAGCCAGGAG |
| DPCH003 | SEQ ID NO: 142 | ATGGCACTAGTAAACTGAGC |
| DPCH004 | SEQ ID NO: 143 | ATCCATATAACTGAAAGCCAA |
| DPCH005 | SEQ ID NO: 144 | ACCACATCATCCATATAACTGAA |
| DPCHRAS001B | SEQ ID NO: 145 | D-LYS-O-O-TTGCCCACACCGCCGGC |
| DPCHRAS002 | SEQ ID NO: 146 | D-LYS-O-O-TCTTGCCCACACCGCC |
| DPCHRAS003 | SEQ ID NO: 147 | D-LYS-O-O-TACTCCTCCTGGCCGGC |
| DPCJ001 | SEQ ID NO: 148 | CGTCTCCACAGACACATACTCCA |
| DPCJ002B | SEQ ID NO: 149 | CGTCTCCACAGACACATACTCCA-O-D-LYS |
| DPCK001B | SEQ ID NO: 150 | GCCTACGCCACCAGCTCCAAC-O-D-LYS |
| DPCK001B2 | SEQ ID NO: 151 | GCCTACGCCACCAGCTCCAAC-O-O-D-LYS |
| DPCK001C | SEQ ID NO: 152 | CTACGCCACCAGCTCCAACTACCA |
| DPCK001C2 | SEQ ID NO: 153 | CTACGCCACCAGCTCCAACTACCA-O-D-LYS |
| DPCK002 | SEQ ID NO: 154 | TCTTGCCTACGCCACCAGCTCCA |
| DPCK003 | SEQ ID NO: 155 | TGTACTCCTCTTGACCTGCTGTG |
| DPCK003B | SEQ ID NO: 156 | D-LYS-O-TGTACTCCTCTTGACCTGCTGTG |
| DPCK004 | SEQ ID NO: 157 | NH(2)-GGCAAATCACATTTATTTCCTAC-CONH(2) |
| DPCK004B | SEQ ID NO: 158 | D-LYS-O-GGCAAATCACATTTATTTCCTAC |
| DPCK005B | SEQ ID NO: 159 | D-LYS-O-TGTCTTGTCTTTGCTGATGTTTC |
| DPCK005 | SEQ ID NO: 160 | TGTCTTGTCTTTGCTGATGTTTC |
| DPCK005C | SEQ ID NO: 161 | D-LYS-O-TGTCTTGTCTTTGCTGATGTTTC |
| DPCK006 | SEQ ID NO: 162 | NH(2)-CTCTTGACCTGCTGTGTCGAG-CONH(2) |
| DPCN001 | SEQ ID NO: 163 | TCCCAACACCACCTGCTCCAA |
| DPCN001B | SEQ ID NO: 164 | D-LYS-O-CAACACCACCTGCTCCAACCACCAC |
| DPCN002 | SEQ ID NO: 165 | CTTTTCCCAACACCACCTGCTCC |
| DPCN002B | SEQ ID NO: 166 | D-LYS-O-TGCGCTTTTCCCAACACCACCTGCT |
| DPCN003B | SEQ ID NO: 167 | GGCACTGTACTCTTCTTGTCCAG |
| DPCN004B | SEQ ID NO: 168 | D-LYS-O-TCTGGTCTTGGCTGAGGTTTC |
| DPCN006 | SEQ ID NO: 169 | NH(2)-GGCAAATCACACTTGTTTCCCAC- |
| | | CONH(2) |
| DPCN006B | SEQ ID NO: 170 | D-LYS-O-GGCAAATCACACTTGTTTCCCAC |
| DPCN007 | SEQ ID NO: 171 | NH(2)-TTCTTGTCCAGCTGTATCCAGTATG- |
| | | CONH(2) |
| DPCPKA003B | SEQ ID NO: 172 | D-LYS-O-AGATCCTCTCTCTGAAATCAC |
| DPCPKA004 | SEQ ID NO: 173 | D-LYS-O-TCTTTCTCCTGCTCAGTGATTTCA |
| DPCPKA005 | SEQ ID NO: 174 | D-LYS-O-AATGATGCACATCATGGTGGCTG |
| NRASN003C | SEQ ID NO: 175 | D-LYS-O-GGCACTGTACTCTTCTTGTCCAG |
| QMDXNA001 | SEQ ID NO: 176 | NH(2)-O-TTCATCAACCGCACTCTGTTTATCTC |
| QMDXNA002 | SEQ ID NO: 177 | NH(2)-O-TGGCGACGACAATGGACCCAATTAT |
| QMDXNA003 | SEQ ID NO: 178 | NH(2)-O-AGATGTAGTTAGCAATCGGTCCTTGTT |
| | | GTA |
| QMDXNA004 | SEQ ID NO: 179 | NH(2)-O-GGGTAATTGAGGTAACGTAGGTATCAA |
| | | GAT |
| QMDXNA005 | SEQ ID NO: 180 | NH(2)-O-TACTATCGACTGACATGAGGCTTGTGT |
| XNADE001 | SEQ ID NO: 181 | D-LYS-O-AGTCCGACGATCTGGAATTC |
| XNADE002 | SEQ ID NO: 182 | D-LYS-O-ACTGGAGTTCAGACGTGTG |
| XNADE003 | SEQ ID NO: 183 | D-LYS-O-CTCTTCCGATCAGATCGGAA |
| XNADE003b | SEQ ID NO: 184 | D-LYS-O-CTCTTCCGATCAGATCGGAAG |
| XNAFGFR001 | SEQ ID NO: 185 | D-LYS-O-O-AGCGCTCCCCGCACC |
| XNAFGFR001 | SEQ ID NO: 186 | D-LYS-O-O-AGCGCTCCCCGCACC |
| XNAFGFR002 | SEQ ID NO: 187 | D-LYS-O-GGGGAGCGCTCTGT-O-TTTTT |
| XNAFGFR003 | SEQ ID NO: 188 | D-LYS-O-O-AGCGCTCCCCGCACC-O-TTTTTT |
| XNAFGFR004 | SEQ ID NO: 189 | D-LYS-O-TGCATACACACTGCCCGCCT |

Other sequences of interest in connection with the invention include the following exons:

BRAF Ex 15 NCBI NG_007873.3; DNA; Wildtype
SEQ ID NO. 190
TAGAAATTAG ATCTCTTACC TAAACTCTTC ATAATGCTTG CTCTGATAGG AAAATGAGAT
CTACTGTTTT CCTTTACTTA CTACACCTCA GATATATTTC TTCATGAAGA CCTCACAGTA
AAAATAGGTG ATTTTGGTCT AGCTACAGTG AAATCTCGAT GGAGTGGGTC CCATCAGTTT
GAACAGTTGT CTGGATCCAT TTTGTGGATG GTAAGAATTG AGGCTATTTT TCCACTGATT
AAATTTTTGG CCCTGAGATG CTGCTGAGTT ACTAGAAAGT CATTGAAGGT CTCAACTATA
GTATTTTCAT AGTTCCCAGT ATTCACAAAA ATCAGTGTTC TTATTTTTTA TGTAAATAGA
EGFR Ex18 GeneGenBank: AF288738.1 NCBI NM_005228.3;
DNA; Wildtype
SEQ ID NO. 191
TAGAGAAGGC GTACATTTGT CCTTCCAAAT GAGCTGGCAA GTGCCGTGTC CTGGCACCCA
AGCCCATGCC GTGGCTGCTG GTCCCCCTGC TGGGCCATGT CTGGCACTGC TTTCCAGCAT
GGTGAGGGCT GAGGTGACCC TTGTCTCTGT GTTCTTGTCC CCCCCAGCTT GTGGAGCCTC
TTACACCCAG TGGAGAAGCT CCCAACCAAG CTCTCTTGAG GATCTTGAAG GAAACTGAAT
TCAAAAAGAT CAAAGTGCTG GGCTCCGGTG CGTTCGGCAC GGTGTATAAG GTAAGGTCCC
TGGCACAGGC CTCTGGGCTG GGCCGCAGGG CCTCTCATGG TCTGGTGGGG AGCCCAGAGT
CCTTGCAAGC TGTATATTTC CATCATCTAC TTTACTCTTT GTTTCACTGA GTGTTTGGGA
AACTCCAGTG TTTTTCCCAA GTTATTGAGA GGAAATCTTT TATAACCACA GTAATCAGTG
EGFR Ex19 GeneGenBank: AF288738.1 NCBI NM_005228.3;
DNA; Wildtype
SEQ ID NO. 192
AGCCCAACAG CTGCAGGGCT GCGGGGGCGT CACAGCCCCC AGCAATATCA GCCTTAGGTG
CGGCTCCACA GCCCCAGTGT CCCTCACCTT CGGGGTGCAT CGCTGGTAAC ATCCACCCAG
ATCACTGGGC AGCATGTGGC ACCATCTCAC AATTGCCAGT TAACGTCTTC CTTCTCTCTC
TGTCATAGGG ACTCTGGATC CCAGAAGGTG AGAAAGTTAA AATTCCCGTC GCTATCAAGG
AATTAAGAGA AGCAACATCT CCGAAAGCCA ACAAGGAAAT CCTCGATGTG AGTTTCTGCT
TTGCTGTGTG GGGGTCCATG GCTCTGAACC TCAGGCCCAC CTTTTCTCAT GTCTGGCAGC
TGCTCTGCTC TAGACCCTGC TCATCTCCAC ATCCTAAATG TTCACTTTCT ATGTCTTTCC
EGFR Ex20 GeneGenBank: AF288738.1 NCBI NM_005228.3;
DNA; Wildtype
SEQ ID NO. 193
AAAATTCCCG TCGCTATCAA GGAATTAAGA GAAGCAACAT CTCCGAAAGC CAACAAGGAA
ATCCTCGATG AAGCCTACGT GATGGCCAGC GTGGACAACC CCCACGTGTG CCGCCTGCTG
GGCATCTGCC TCACCTCCAC CGTGCAGCTCATCACGCAGC TCATGCCCTT CGGCTGCCTC
CTGGACTATG TCCGGGAACA CAAAGACAAT ATTGGCTCCC AGTACCTGCT CAACTGGTGT
GTGCAGATCG CAAAGGGCAT GAACTACTTG GAGGACCGTC GCTTGGTGCA CCGCGACCTG
GCAGCCAGGA ACGTACTGGT GAAAACACCG CAGCATGTCA AGATCACAGA TTTTGGGCTG
GCCAAACTGC TGGGTGCGGA AGAGAAAGAA TACCATGCAG AAGGAGGCAA AGTGCCTATC
AAGTGGATGG CATTGGAATC AATTTTACAC AGAATCTATA CCCACCAGAG TGATGTCTGG
EGFR Ex21 GeneGenBank: AF288738.1 NCBI NM_005228.3;
DNA; Wildtyp
SEQ ID NO. 194
GGCATCTGCC TCACCTCCAC CGTGCAGCTC ATCACGCAGC TCATGCCCTT CGGCTGCCTC
CTGGACTATG TCCGGGAACA CAAAGACAAT ATTGGCTCCC AGTACCTGCT CAACTGGTGT
GTGCAGATCG CAAAGGGCAT GAACTACTTG GAGGACCGTC GCTTGGTGCA CCGCGACCTG
GCAGCCAGGA ACGTACTGGT GAAAACACCG CAGCATGTCA AGATCACAGA TTTTGGGCTG
GCCAAACTGC TGGGTGCGGA AGAGAAAGAA TACCATGCAG AAGGAGGCAA AGTGCCTATC
AAGTGGATGG CATTGGAATC AATTTTACAC AGAATCTATA CCCACCAGAG TGATGTCTGG
AGCTACGGGG TGACCGTTTG GGAGTTGATG ACCTTTGGAT CCAAGCCATA TGACGGAATC
HRAS Ex3 NCBI Reference Sequence: NG_007666.1;
DNA; Wildtype
SEQ ID NO. 195
CTGCAGGATT CCTACCGGAA GCAGGTGGTC ATTGATGGGG AGACGTGCCT GTTGGACATC
CTGGATACCG CCGGCCAGGA GGAGTACAGC GCCATGCGGG ACCAGTACAT GCGCACCGGG
GAGGGCTTCC TGTGTGTGTT TGCCATCAAC AACACCAAGT CTTTTGAGGA CATCCACCAG
TACAGGTGAA CCCCGTGAGG CTGGCCCGGG AGCCCACGCC GCACAGGTGG GGCCAGGCC
JAK2 NCBI Reference Sequence: NG_009904.1;
DNA; wildtype
SEQ ID NO. 196
CTGACATCTACCTCTAGTTGTACTTCTGTCCTCTATTTCAGGTGTTATGGGTCAAGCCTGTTTGACTGGC
ATTATTCATGATTCCTGTACCACTCTTGCTCTCTCTCACTTTGATCTCCATATTCCAGGCTTACACAGGG
GTTTCCTCAGAACGTTGATGGCAGTTGCAGGTCCATATAAAGGGACCAAAGCACATTGTATCCTCATC
TAGTCATGCTGAAAGTAGGAGAAAGTGCATCTTTATTATGGCAGAGAGAATTTTCTGAACTATTTATG
GACAACAGTCAAACAACAATTCTTTGTACTTTTTTTTTTCCTTAGTCTTTCTTTGAAGCAGCAAGTATGA
TGAGCAAGCTTTCTCACAAGCATTTGGTTTTAAATTATGGAGTATGTGTCTGTGGAGACGAGAGTAAG
TAAAACTACAGGCTTTCTAATGCCTTTCTCAGAGCATCTGTTTTTGTTTATATAGAAAATTCAGTTTCAG
GATCACAGCTAGGTGTCAGTGTAAACTATAATTTAACAGGAGTTAAGTATTTTTGAAACTGAAAACAC
TGTAGGACTATTCAGTTATATCTTGTGAAAAAGGAAAGCAATGAAGTTAAAAGTAGAAGGTTACAATG
CCCAAACAATAGAGTATTATAGTAAACAAATGTCTATAAAACATTTTGTGTTCATGATAGCAAAAGAG
ATTATGGCAGGTTCAACATAACATTGGAATAACTGGCCTTTTCAGTACAAACTTATCTGGAATTATGAA
GACAAAGCATA
KRAS Ex2 NCBI Reference Sequence: NG_007524.1;
DNA; wildtype
SEQ ID NO. 197
GGTACTGGTG GAGTATTTGA TAGTGTATTA ACCTTATGTG TGACATGTTC TAATATAGTC
ACATTTTCAT TATTTTTATT ATAAGGCCTG CTGAAAATGA CTGAATATAA ACTTGTGGTA
GTTGGAGCTG GTGGCGTAGG CAAGAGTGCC TTGACGATAC AGCTAATTCA GAATCATTTT
GTGGACGAAT ATGATCCAAC AATAGAGGTA AATCTTGTTT TAATATGCAT ATTACTGGTG
KRAS Ex3 NCBI Reference Sequence: NG_007524.1;
DNA; wildtype
SEQ ID NO. 198
CTTCTCAGGA TTCCTACAGG AAGCAAGTAG TAATTGATGG AGAAACCTGT CTCTTGGATA
TTCTCGACAC AGCAGGTCAA GAGGAGTACA GTGCAATGAG GGACCAGTAC ATGAGGACTG
GGGAGGGCTT TCTTTGTGTA TTTGCCATAA ATAATACTAA ATCATTTGAA GATATTCACC
ATTATAGGTG GGTTTAAATT GAATATAATA AGCTGACATT AAGGAGTAAT TATAGTTTTT
KRAS Ex4 NCBI Reference Sequence: NG_007524.1;
DNA; wildtype
SEQ ID NO. 199
GTGCTATAAC TTTTTTTTCT TTCCCAGAGA ACAAATTAAA AGAGTTAAGG ACTCTGAAGA
TGTACCTATG GTCCTAGTAG GAAATAAATG TGATTTGCCT TCTAGAACAG TAGACACAAA
ACAGGCTCAG GACTTAGCAA GAAGTTATGG AATTCCTTTT ATTGAAACAT CAGCAAAGAC
AAGACAGGTA AGTAACACTG AAATAAATAC AGATCTGTTT TCTGCAAAAT CATAACTGTT
ATGTCATTTA ATATATCAGT TTTTCTCTCA ATTATGCTAT ACTAGGAAAT AAAACAATAT
KRAS Ex5 NCBI Reference Sequence: NG_007524.1;
DNA; wildtype
SEQ ID NO. 200
AATGCAACAG ACTTTAAAGA AGTTGTGTTT TACAATGCAG AGAGTGGAGG ATGCTTTTTA
TACATTGGTG AGGGAGATCC GACAATACAG ATTGAAAAAA ATCAGCAAAG AAGAAAAGAC
TCCTGGCTGT GTGAAAATTA AAAAATGCAT TATAATGTAA TCTGGTAAGT TTAAGTTCAG
NRAS Exon 2 NCBI Reference Sequence: NG_007572.1;
DNA; wildtype
SEQ ID NO. 201
GTGTTTTTGC GTTCTCTAGT CACTTTAAGA ACCAAATGGA AGGTCACACT AGGGTTTTCA
TTTCCATTGA TTATAGAAAG CTTTAAAGTA CTGTAGATGT GGCTCGCCAA TTAACCCTGA
TTACTGGTTT CCAACAGGTT CTTGCTGGTG TGAAATGACT GAGTACAAAC TGGTGGTGGT
TGGAGCAGGT GGTGTTGGGA AAAGCGCACT GACAATCCAG CTAATCCAGA ACCACTTTGT
AGATGAATAT GATCCCACCA TAGAGGTGAG GCCCAGTGGT AGCCCGCTGA CCTGATCCTG
TCTCTCACTT GTCGGATCAT CTTTACCCAT ATTCTGTATT AAAGGAATAA GAGGAGAGAA
AGTAAAAAGT TATTTTGGGT ATACATTCAG TTATGCAATA AGCTTAACGT GTTTATAGAG
AACAGTTCAT TTTTATTAGC TGCTGAAGTT TCTAAAACCT GTCCAGTTTT TAACAGTTCT
NRAS Exon 3 NCBI Reference Sequence: NG_007572.1;
DNA; wildtype
SEQ ID NO. 202
TGGGCTTGAA TAGTTAGATG CTTATTTAAC CTTGGCAATA GCATTGCATT CCCTGTGGTT
TTTAATAAAA ATTGAACTTC CCTCCCTCCC TGCCCCCTTA CCCTCCACAC CCCCAGGATT
CTTACAGAAA ACAAGTGGTT ATAGATGGTG AAACCTGTTT GTTGGACATA CTGGATACAG
CTGGACAAGA AGAGTACAGT GCCATGAGAG ACCAATACAT GAGGACAGGC GAAGGCTTCC
TCTGTGTATT TGCCATCAAT AATAGCAAGT CATTTGCGGA TATTAACCTC TACAGGTACT
AGGAGCATTA TTTTCTCTGA AAGGATGATC TTTGTGTTCT GAATCTTTAT GGGGAAATGA
GGTTACCACA CTAGGGAAGA TAGAGCTTTT TAATTATGGG AAGAGTTGGT TTTAGGTTGT
TTGACATTGA GAATCTAGGG TAATTACTGA AAGTTAATAC TGGAATTTAT TTTACATAAT
NRAS Exon 4 NCBI Reference Sequence: NG_007572.1;
DNA; wildtype
SEQ ID NO. 203
TGGATACAGC TGGACAAGAA GAGTACAGTG CCATGAGAGA CCAATACATG AGGACAGGCG
AAGGCTTCCT CTGTGTATTT GCCATCAATA ATAGCAAGTC ATTTGCGGAT ATTAACCTCT
ACAGGGAGCA GATTAAGCGA GTAAAAGACT CGGATGATGT ACCTATGGTG CTAGTGGGAA
ACAAGTGTGA TTTGCCAACA AGGACAGTTG ATACAAAACA AGCCCACGAA CTGGCCAAGA
GTTACGGGAT TCCATTCATT GAAACCTCAG CCAAGACCAG ACAGGGTGTT GAAGATGCTT
TTTACACACTGGTAAGAGAA ATACGCCAGT ACCGAATGAA AAAACTCAAC AGCAGTGATG
ATGGGACTCA GGGTTGTATG GGATTGCCAT GTGTGGTGAT GTAACAAGAT ACTTTTAAAG
PIK3CA Ex9 NCBI Reference Sequence: NG_012113.2;
DNA; wildtype
SEQ ID NO. 204
TGTAAAATTT ATTGAAAATG TATTTGCTTT TTCTGTAAAT CATCTGTGAA TCCAGAGGGG
AAAAATATGA CAAAGAAAGC TATATAAGAT ATTATTTTAT TTTACAGAGT AACAGACTAG
CTAGAGACAA TGAATTAAGG GAAAATGACA AAGAACAGCT CAAAGCAATT TCTACACGAG
ATCCTCTCTC TGAAATCACT GAGCAGGAGA AAGATTTTCT ATGGAGTCAC AGGTAAGTGC
TAAAATGGAG ATTCTCTGTT TCTTTTTCTT TATTACAGAA AAAATAACTG AATTTGGCTG
ATCTCAGCAT GTTTTTACCA TACCTATTGG AATAAATAAA GCAGAATTTA CATGATTTTT
PIK3CA Ex20NCBI Reference Sequence: NG_012113.2;
DNA; wildtype
SEQ ID NO. 205
TAGCTATTCG ACAGCATGCC AATCTCTTCA TAAATCTTTT CTCAATGATG CTTGGCTCTG
GAATGCCAGA ACTACAATCT TTTGATGACA TTGCATACAT TCGAAAGACC CTAGCCTTAG
ATAAAACTGA GCAAGAGGCT TTGGAGTATT TCATGAAACA AATGAATGAT GCACATCATG
GTGGCTGGAC AACAAAAATG GATTGGATCT TCCACACAAT TAAACAGCAT GCATTGAACT
GAAAAGATAA CTGAGAAAAT GAAAGCTCAC TCTGGATTCC ACACTGCACT GTTAATAACT
PIK3CA Ex16 NCBI Reference Sequence: NG_012113.2;
DNA; wildtype
SEQ ID NO. 206
GTTGTAAATCTTTGTAACACTTCAAAAAGCTATATTGTATTTATATTTTAAAATAAATTTCAGGGTAAA
ATAATAATAAAGCAAAGGTACCTAGTAAAGTTTTTAACTATTTTAAAGGCTTGAAGAGTGTCGAATTA
TGTCCTCTGCAAAAAGGCCACTGTGGTTGAATTGGGAGAACCCAGACATCATGTCAGAGTTACTGTTT
CAGAACAATGAGATCATCTTTAAAAATGGGGATGGTAAGGAAGAGTATTAATGAGCTTATGATGCATG
AATTTAGCTATCTTTTTATACACAGGATATTTATGAACCATGAAAACTACTGAAAGCCATTTAAGGAAT
ATACACATGTGATAAAATATGTAATATTTATCAGATGTCTTGACCTTTGAAATATGCATGTATAATCAA
TGAAAAGAAAAGAAGTACTAGGTTTAGATCAGAAGTCCTGAAATCAGTTTTTTGTTTTTTCTTTTTCCT
GTTCCCTGCC

All literature and similar materials cited in this application including, but not limited to, patents, patent applications, articles, books, treatises, and internet web pages, regardless of the format of such literature and similar materials, are expressly incorporated by reference in their entirety for any purpose as if they were entirely denoted. In the event that one or more of the incorporated literature and similar materials defines or uses a term in such a way that it contradicts that term's definition in this application, this application controls.

Although the foregoing description contains many specifics, these should not be construed as limiting the scope of the present invention, but merely as providing illustrations of some of the presently preferred embodiments. Similarly, other embodiments may be devised without departing from the spirit or scope of the present invention. Features from different embodiments may be employed in combination. The scope of the invention is, therefore, indicated and limited only by the appended claims and their legal equivalents rather than by the foregoing description. All additions, deletions and modifications to the invention as disclosed herein which fall within the meaning and scope of the claims are to be embraced thereby.

What is claimed is:

1. A method for enriching a target polynucleotide sequence containing a genetic variation said method comprising:
(a) providing two primers targeted to said target polynucleotide sequence;
(b) providing a target specific xenonucleic acid clamp oligomer specific for a wildtype polynucleotide sequence;
(c) generating multiple amplicons using PCR under specific temperature cycling conditions; and
(d) detecting said amplicons.

2. The method of claim 1, wherein said detection employs oligonucleotide probes specific for hybridization of variant polynucleotide amplicon sequences.

3. The method of claim 1, wherein the variant target sequence is in a gene selected from the group consisting of: KRAS, BRAF, EGFR, TP53, JAK2, NPM1, and PCA3.

4. A method for enriching multiple target polynucleotide sequences containing a genetic variation said method comprising:
(a) providing a library of amplifying primers targeted to said multiple target polynucleotide sequence;
(b) providing a library of target specific xenonucleic acid clamp oligomer specific for multiple wildtype polynucleotide sequences;
(c) generating multiple amplicons using PCR under specific temperature cycling conditions; and
(d) detecting said amplicons.

5. The method of claim 3, wherein said detection employs oligonucleotide probes specific for hybridization of variant polynucleotide amplicon sequences.

6. A method for conducting a minimally invasive biopsy in a mammalian subject suspected of a having a neoplastic disease, said method comprising:
(a) sampling of target polynucleotides derived from said mammalian subject;
(b) providing a library of amplifying primers targeted to said multiple target poly-nucleotide sequence;
(c) providing a library of target specific xenonucleic acid clamp oligomer specific for multiple wildtype polynucleotide sequences;
(d) generating multiple amplicons using PCR under specific temperature cycling conditions; and
(e) detecting said amplicons.

7. The method of claim 5, wherein said sampled target polynucleotides are sampled from cells derived from said mammalian subject.

8. The method of claim 5, wherein said sampled target polynucleotides are sampled from free circulating cell free polynucleotides derived from said mammalian subject.

9. The method of claim 4, which includes using multiple XNA clamp probes and amplifying primers targeted to multiple polynucleotide sequences


SEQUENCE LISTING (206 sequences)

SEQ ID NO: 1 - DNA, 13 bp, synthetic construct
  source 1..13: mol_type = other DNA
agctgctggcgta

SEQ ID NO: 2 - DNA, 13 bp, synthetic construct
  source 1..13: mol_type = other DNA
agctcgtggcgta

SEQ ID NO: 3 - DNA, 13 bp, synthetic construct
  source 1..13: mol_type = other DNA
agctgatggcgta

SEQ ID NO: 4 - DNA, 13 bp, synthetic construct
  source 1..13: mol_type = other DNA
agcttgtggcgta

SEQ ID NO: 5 - DNA, 13 bp, synthetic construct
  source 1..13: mol_type = other DNA
gagctattggcgt

SEQ ID NO: 6 - DNA, 13 bp, synthetic construct
  source 1..13: mol_type = other DNA
gagctcttggcgt

SEQ ID NO: 7 - DNA, 13 bp, synthetic construct
  source 1..13: mol_type = other DNA
agctagtggcgta

SEQ ID NO: 8 - DNA, 13 bp, synthetic construct
  source 1..13: mol_type = other DNA
agctgttggcgta

SEQ ID NO: 9 - DNA, 13 bp, synthetic construct
  source 1..13: mol_type = other DNA
tggttgcgtaggc

SEQ ID NO: 10 - DNA, 13 bp, synthetic construct
  source 1..13: mol_type = other DNA
tggtgacgtaggc

SEQ ID NO: 11 - DNA, 13 bp, synthetic construct
  source 1..13: mol_type = other DNA
tggtgccgtaggc

SEQ ID NO: 12 - DNA, 13 bp, synthetic construct
  source 1..13: mol_type = other DNA
tggtgtcgtaggc

SEQ ID NO: 13 - DNA, 13 bp, synthetic construct
  source 1..13: mol_type = other DNA
tggtagcgtaggc

SEQ ID NO: 14 - DNA, 13 bp, synthetic construct
  source 1..13: mol_type = other DNA
tggtcgcgtaggc

SEQ ID NO: 15 - DNA, 13 bp, synthetic construct
  source 1..13: mol_type = other DNA
ggagacggtctgg

SEQ ID NO: 16 - DNA, 13 bp, synthetic construct
  source 1..13: mol_type = other DNA
ggagacgctctgg

SEQ ID NO: 17 - DNA, 13 bp, synthetic construct
  source 1..13: mol_type = other DNA
ctggtggcgtagg

SEQ ID NO: 18 - DNA, 13 bp, synthetic construct
  source 1..13: mol_type = other DNA
aaggcctgctgaa

SEQ ID NO: 19 - DNA, 24 bp, synthetic construct
  source 1..24: mol_type = other DNA
gtactggtggagtatttgatagtg

SEQ ID NO: 20 - DNA, 23 bp, synthetic construct
  source 1..23: mol_type = other DNA
atcgtcaaggcactcttgcctac

SEQ ID NO: 21 - DNA, 27 bp, synthetic construct
  source 1..27: mol_type = other DNA
cgaaagaccctagccttagataaaact

SEQ ID NO: 22 - DNA, 25 bp, synthetic construct
  source 1..25: mol_type = other DNA
attgtgtggaagatccaatccattt

SEQ ID NO: 23 - DNA, 32 bp, synthetic construct
  source 1..32: mol_type = other DNA
acgttggatgtgtaccatacctgtctggtctt

SEQ ID NO: 24 - DNA, 48 bp, synthetic construct
  source 1..48: mol_type = other DNA
gttttcccagtcacgacacgttggatgcagccaggaacgtactggtga

SEQ ID NO: 25 - DNA, 23 bp, synthetic construct
  source 1..23: mol_type = other DNA
  misc_feature 1..23: note = Biotin modified
ctccagatctcagtaaggtacgg

SEQ ID NO: 26 - DNA, 22 bp, synthetic construct
  source 1..22: mol_type = other DNA
  misc_feature 1..22: note = Biotin modified
tgagggagatccgacaatacag

SEQ ID NO: 27 - DNA, 31 bp, synthetic construct
  source 1..31: mol_type = other DNA
acagtaaaaataggtgattttggtctagcta

SEQ ID NO: 28 - DNA, 58 bp, synthetic construct
  source 1..58: mol_type = other DNA
gttttcccagtcacgacacgttggatgacagtaaaaataggtgattttggtctagcta

SEQ ID NO: 29 - DNA, 24 bp, synthetic construct
  source 1..24: mol_type = other DNA
catccacaaaatggatccagacaa

SEQ ID NO: 30 - DNA, 51 bp, synthetic construct
  source 1..51: mol_type = other DNA
caggaaacagctatgacacgttggatgcatccacaaaatggatccagacaa

SEQ ID NO: 31 - DNA, 23 bp, synthetic construct
  source 1..23: mol_type = other DNA
ctccagatctcagtaaggtacgg

SEQ ID NO: 32 - DNA, 21 bp, synthetic construct
  source 1..21: mol_type = other DNA
gggaaagagtggtctctcatc

SEQ ID NO: 33 - DNA, 28 bp, synthetic construct
  source 1..28: mol_type = other DNA
acgttggatgtccaccgtgcagctcatc

SEQ ID NO: 34 - DNA, 41 bp, synthetic construct
  source 1..41: mol_type = other DNA
gttttcccagtcacgacacgttggatgtccaccgtgcagct

SEQ ID NO: 35 - DNA, 30 bp, synthetic construct
  source 1..30: mol_type = other DNA
acgttggatggtctttgtgttcccggacat

SEQ ID NO: 36 - DNA, 41 bp, synthetic construct
  source 1..41: mol_type = other DNA
caggaaacagctatgacacgttggatggtctttgtgttccc

SEQ ID NO: 37 - DNA, 53 bp, synthetic construct
  source 1..53: mol_type = other DNA
gttttcccagtcacgacacgttggatgctctctgtcatagggactctggatcc

SEQ ID NO: 38 - DNA, 26 bp, synthetic construct
  source 1..26: mol_type = other DNA
ctctctgtcatagggactctggatcc

SEQ ID NO: 39 - DNA, 24 bp, synthetic construct
  source 1..24: mol_type = other DNA
agcaaagcagaaactcacatcgag

SEQ ID NO: 40 - DNA, 51 bp, synthetic construct
  source 1..51: mol_type = other DNA
caggaaacagctatgacacgttggatgagcaaagcagaaactcacatcgag

SEQ ID NO: 41 - DNA, 49 bp, synthetic construct
  source 1..49: mol_type = other DNA
gttttcccagtcacgacacgttggatggctcccaaccaagctctcttga

SEQ ID NO: 42 - DNA, 22 bp, synthetic construct
  source 1..22: mol_type = other DNA
gctcccaaccaagctctcttga

SEQ ID NO: 43 - DNA, 25 bp, synthetic construct
  source 1..25: mol_type = other DNA
ctgtgccagggaccttaccttatac

SEQ ID NO: 44 - DNA, 52 bp, synthetic construct
  source 1..52: mol_type = other DNA
caggaaacagctatgacacgttggatgctgtgccagggaccttaccttatac

SEQ ID NO: 45 - DNA, 23 bp, synthetic construct
  source 1..23: mol_type = other DNA
tttgccaaggcacgagtaacaag

SEQ ID NO: 46 - DNA, 23 bp, synthetic construct
  source 1..23: mol_type = other DNA
cccaaggaccacctcacagttat

SEQ ID NO: 47 - DNA, 26 bp, synthetic construct
  source 1..26: mol_type = other DNA
ttaactgcagatgcacatcattacct

SEQ ID NO: 48 - DNA, 23 bp, synthetic construct
  source 1..23: mol_type = other DNA
ggactctgaagatgtacctatgg

SEQ ID NO: 49 - DNA, 50 bp, synthetic construct
  source 1..50: mol_type = other DNA
gttttcccagtcacgacacgttggatgggactctgaagatgtacctatgg

SEQ ID NO: 50 - DNA, 20 bp, synthetic construct
  source 1..20: mol_type = other DNA
gctaagtcctgagcctgttt

SEQ ID NO: 51 - DNA, 47 bp, synthetic construct
  source 1..47: mol_type = other DNA
caggaaacagctatgacacgttggatggctaagtcctgagcctgttt

SEQ ID NO: 52 - DNA, 20 bp, synthetic construct
  source 1..20: mol_type = other DNA
acacaaaacaggctcaggac

SEQ ID NO: 53 - DNA, 47 bp, synthetic construct
  source 1..47: mol_type = other DNA
gttttcccagtcacgacacgttggatgacacaaaacaggctcaggac

SEQ ID NO: 54 - DNA, 25 bp, synthetic construct
  source 1..25: mol_type = other DNA
cagtgttacttacctgtcttgtctt

SEQ ID NO: 55 - DNA, 52 bp, synthetic construct
  source 1..52: mol_type = other DNA
caggaaacagctatgacacgttggatgcagtgttacttacctgtcttgtctt

SEQ ID NO: 56 - DNA, 21 bp, synthetic construct
  source 1..21: mol_type = other DNA
aaggcctgctgaaaatgactg

SEQ ID NO: 57 - DNA, 48 bp, synthetic construct
  source 1..48: mol_type = other DNA
gttttcccagtcacgacacgttggatgaaggcctgctgaaaatgactg

SEQ ID NO: 58 - DNA, 48 bp, synthetic construct
  source 1..48: mol_type = other DNA
caggaaacagctatgacacgttggatgtcaaggcactcttgcctacgc

SEQ ID NO: 59 - DNA, 22 bp, synthetic construct
  source 1..22: mol_type = other DNA
acttgtggtagttggagctggt

SEQ ID NO: 60 - DNA, 49 bp, synthetic construct
  source 1..49: mol_type = other DNA
gttttcccagtcacgacacgttggatgacttgtggtagttggagctggt

SEQ ID NO: 61 - DNA, 25 bp, synthetic construct
  source 1..25: mol_type = other DNA
tcatgaaaatggtcagagaaacctt

SEQ ID NO: 62 - DNA, 49 bp, synthetic construct
  source 1..49: mol_type = other DNA
caggaaacagctatgacacgttggatgacttgtggtagttggagctggt

SEQ ID NO: 63 - DNA, 23 bp, synthetic construct
  source 1..23: mol_type = other DNA
attgcactgtactcctcttgacc

SEQ ID NO: 64 - DNA, 50 bp, synthetic construct
  source 1..50: mol_type = other DNA
caggaaacagctatgacacgttggatgattgcactgtactcctcttgacc

SEQ ID NO: 65 - DNA, 27 bp, synthetic construct
  source 1..27: mol_type = other DNA
ctcttggatattctcgacacagcaggt

SEQ ID NO: 66 - DNA, 54 bp, synthetic construct
  source 1..54: mol_type = other DNA
gttttcccagtcacgacacgttggatgctcttggatattctcgacacagcaggt

SEQ ID NO: 67 - DNA, 20 bp, synthetic construct
  source 1..20: mol_type = other DNA
ccagactgtgtttctccctt

SEQ ID NO: 68 - DNA, 47 bp, synthetic construct
  source 1..47: mol_type = other DNA
gttttcccagtcacgacacgttggatgccagactgtgtttctccctt

SEQ ID NO: 69 - DNA, 22 bp, synthetic construct
  source 1..22: mol_type = other DNA
tgagggagatccgacaatacag

SEQ ID NO: 70 - DNA, 25 bp, synthetic construct
  source 1..25: mol_type = other DNA
tctgccaaaattaatgtgctgaact

SEQ ID NO: 71 - DNA, 18 bp, synthetic construct
  source 1..18: mol_type = other DNA
ttctcttccgcacccagc

SEQ ID NO: 72 - DNA, 45 bp, synthetic construct
  source 1..45: mol_type = other DNA
caggaaacagctatgacacgttggatgttctcttccgcacccagc

SEQ ID NO: 73 - DNA, 51 bp, synthetic construct
  source 1..51: mol_type = other DNA
gttttcccagtcacgacacgttggatgtgaaaacaccgcagcatgtcaaga

SEQ ID NO: 74 - DNA, 24 bp, synthetic construct
  source 1..24: mol_type = other DNA
tgaaaacaccgcagcatgtcaaga

SEQ ID NO: 75 - DNA, 24 bp, synthetic construct
  source 1..24: mol_type = other DNA
ccttactttgcctccttctgcatg

SEQ ID NO: 76 - DNA, 51 bp, synthetic construct
  source 1..51: mol_type = other DNA
caggaaacagctatgacacgttggatgccttactttgcctccttctgcatg

SEQ ID NO: 77 - DNA, 26 bp, synthetic construct
  source 1..26: mol_type = other DNA
tggtgggatcatattcatctacaaag

SEQ ID NO: 78 - DNA, 26 bp, synthetic construct
  source 1..26: mol_type = other DNA
tggtgggatcatattcatctacaaag

SEQ ID NO: 79 - DNA, 53 bp, synthetic construct
  source 1..53: mol_type = other DNA
caggaaacagctatgacacgttggatgtggtgggatcatattcatctacaaag

SEQ ID NO: 80 - DNA, 27 bp, synthetic construct
  source 1..27: mol_type = other DNA
agtaaaagactcggatgatgtacctat

SEQ ID NO: 81 - DNA, 32 bp, synthetic construct
  source 1..32: mol_type = other DNA
acgttggatgacctatggtgctagtgggaaac

SEQ ID NO: 82 - DNA, 30 bp, synthetic construct
  source 1..30: mol_type = other DNA
acgttggatgtcccgtttttagggagcaga

SEQ ID NO: 83 - DNA, 20 bp, synthetic construct
  source 1..20: mol_type = other DNA
cccgtttttagggagcagat

SEQ ID NO: 84 - DNA, 20 bp, synthetic construct
  source 1..20: mol_type = other DNA
cagttcgtgggcttgttttg

SEQ ID NO: 85 - DNA, 20 bp, synthetic construct
  source 1..20: mol_type = other DNA
cttgcacaaatgctgaaagc

SEQ ID NO: 86 - DNA, 21 bp, synthetic construct
  source 1..21: mol_type = other DNA
aaactggtggtggttggagca

SEQ ID NO: 87 - DNA, 48 bp, synthetic construct
  source 1..48: mol_type = other DNA
gttttcccagtcacgacacgttggatgaaactggtggtggttggagca

SEQ ID NO: 88 - DNA, 19 bp, synthetic construct
  source 1..19: mol_type = other DNA
ggtggtggttggagcaggt

SEQ ID NO: 89 - DNA, 46 bp, synthetic construct
  source 1..46: mol_type = other DNA
gttttcccagtcacgacacgttggatgggtggtggttggagcaggt

SEQ ID NO: 90 - DNA, 22 bp, synthetic construct
  source 1..22: mol_type = other DNA
acacccccaggattcttacaga

SEQ ID NO: 91 - DNA, 49 bp, synthetic construct
  source 1..49: mol_type = other DNA
gttttcccagtcacgacacgttggatgacacccccaggattcttacaga

SEQ ID NO: 92 - DNA, 23 bp, synthetic construct
  source 1..23: mol_type = other DNA
atggcactgtactcttcttgtcc

SEQ ID NO: 93 - DNA, 50 bp, synthetic construct
  source 1..50: mol_type = other DNA
caggaaacagctatgacacgttggatgatggcactgtactcttcttgtcc

SEQ ID NO: 94 - DNA, 25 bp, synthetic construct
  source 1..25: mol_type = other DNA
gttggacatactggatacagctgga

SEQ ID NO: 95 - DNA, 52 bp, synthetic construct
  source 1..52: mol_type = other DNA
gttttcccagtcacgacacgttggatggttggacatactggatacagctgga

SEQ ID NO: 96 - DNA, 20 bp, synthetic construct
  source 1..20: mol_type = other DNA
ccgcaaatgacttgctatta

SEQ ID NO: 97 - DNA, 47 bp, synthetic construct
  source 1..47: mol_type = other DNA
caggaaacagctatgacacgttggatgccgcaaatgacttgctatta

SEQ ID NO: 98 - DNA, 21 bp, synthetic construct
  source 1..21: mol_type = other DNA
acacactggtaagagaaatac

SEQ ID NO: 99 - DNA, 18 bp, synthetic construct
  source 1..18: mol_type = other DNA
ctgagtcccatcatcact

SEQ ID NO: 100 - DNA, 24 bp, synthetic construct
  source 1..24: mol_type = other DNA
atcgagatttcactgtagctagac

SEQ ID NO: 101 - DNA, 19 bp, synthetic construct
  source 1..19: mol_type = other DNA
acttcaggcagcgtcttca

SEQ ID NO: 102 - DNA, 19 bp, synthetic construct
  source 1..19: mol_type = other DNA
tgttcagagcacacttcag

SEQ ID NO: 103 - DNA, 19 bp, synthetic construct
  source 1..19: mol_type = other DNA
ctggtggttgaatttgctg

SEQ ID NO: 104 - DNA, 22 bp, synthetic construct
  source 1..22: mol_type = other DNA
catgagctccagcaggatgaac

SEQ ID NO: 105 - DNA, 19 bp, synthetic construct
  source 1..19: mol_type = other DNA
ccgaagtctccaatcttgg

SEQ ID NO: 106 - DNA, 19 bp, synthetic construct
  source 1..19: mol_type = other DNA
tagatgtctcgggccatcc

SEQ ID NO: 107 - DNA, 15 bp, synthetic construct
  source 1..15: mol_type = other DNA
gggacactctaagat

SEQ ID NO: 108 - DNA, 18 bp, synthetic construct
  source 1..18: mol_type = other DNA
ttctgtcctgggattctc

SEQ ID NO: 109 - DNA, 18 bp, synthetic construct
  source 1..18: mol_type = other DNA
agattttccacttgctgt

SEQ ID NO: 110 - DNA, 25 bp, synthetic construct
  source 1..25: mol_type = other DNA
ccagatgggacactctaagattttc

SEQ ID NO: 111 - DNA, 23 bp, synthetic construct
  source 1..23: mol_type = other DNA
cctttctgtcctgggattctctt

SEQ ID NO: 112 - DNA, 26 bp, synthetic construct
  source 1..26: mol_type = other DNA
gacagattttccacttgctgtgctaa

SEQ ID NO: 113 - DNA, 22 bp, synthetic construct
  source 1..22: mol_type = other DNA
cataaaggacactgtgaaggcc

SEQ ID NO: 114 - DNA, 22 bp, synthetic construct
  source 1..22: mol_type = other DNA
  misc_feature 1..22: note = D-Lysine modified
ggccttcacagtgtcctttatg

SEQ ID NO: 115 - DNA, 22 bp, synthetic construct
  source 1..22: mol_type = other DNA
  misc_feature 1..22: note = D-Lysine modified
cattcttgatgtctctggctag

SEQ ID NO: 116 - DNA, 14 bp, synthetic construct
  source 1..14: mol_type = other DNA
gagcccagcacttt

SEQ ID NO: 117 - DNA, 19 bp, synthetic construct
  source 1..19: mol_type = other DNA
  misc_feature 1..19: note = D-Lysine modified
cggagcccagcactttgat

SEQ ID NO: 118 - DNA, 19 bp, synthetic construct
  source 1..19: mol_type = other DNA
  misc_feature 1..19: note = D-Lysine modified
cggagcccagcactttgat

SEQ ID NO: 119 - DNA, 18 bp, synthetic construct
  source 1..18: mol_type = other DNA
  misc_feature 1..18: note = Amino and carboxamido modified
agatgttgcttctcttaa

SEQ ID NO: 120 - DNA, 18 bp, synthetic construct
  source 1..18: mol_type = other DNA
  misc_feature 1..18: note = D-Lysine modified
agatgttgcttctcttaa

SEQ ID NO: 121 - DNA, 25 bp, synthetic construct
  source 1..25: mol_type = other DNA
  misc_feature 1..25: note = D-Lysine modified
cggagatgttgcttctcttaattcc

SEQ ID NO: 122 - DNA, 16 bp, synthetic construct
  source 1..16: mol_type = other DNA
cagtttggccagccca

SEQ ID NO: 123 - DNA, 16 bp, synthetic construct
  source 1..16: mol_type = other DNA
  misc_feature 1..16: note = D-Lysine modified
cagtttggccagccca

SEQ ID NO: 124 - DNA, 21 bp, synthetic construct
  source 1..21: mol_type = other DNA
tttggccagcccaaaatctgt

SEQ ID NO: 125 - DNA, 18 bp, synthetic construct
  source 1..18: mol_type = other DNA
ggccagcccaaaatctgt

SEQ ID NO: 126 - DNA, 15 bp, synthetic construct
  source 1..15: mol_type = other DNA
acccagcagtttggc

SEQ ID NO: 127 - DNA, 15 bp, synthetic construct
  source 1..15: mol_type = other DNA
acccagcagtttggc

SEQ ID NO: 128 - DNA, 13 bp, synthetic construct
  source 1..13: mol_type = other DNA
gctgcgtgatgag

SEQ ID NO: 129 - DNA, 12 bp, synthetic construct
  source 1..12: mol_type = other DNA
gctgcgtgatga

SEQ ID NO: 130 - DNA, 20 bp, synthetic construct
  source 1..20: mol_type = other DNA
agctcatcacgcagctcatg

SEQ ID NO: 131 - DNA, 22 bp, synthetic construct
  source 1..22: mol_type = other DNA
  misc_feature 1..22: note = D-Lysine modified
cagctcatcacgcagctcatgc

SEQ ID NO: 132 - DNA, 22 bp, synthetic construct
  source 1..22: mol_type = other DNA
  misc_feature 1..22: note = D-Lysine modified
tcatcacgcagctcatgccctt

SEQ ID NO: 133 - DNA, 18 bp, synthetic construct
  source 1..18: mol_type = other DNA
  misc_difference 1..18: note = D-Lysine modified
ctcatcacgcagctcatg

SEQ ID NO: 134 - DNA, 14 bp, synthetic construct
  source 1..14: mol_type = other DNA
  misc_feature 1..14: note = D-Lysine modified
tgagctgcgtgatg

SEQ ID NO: 135 - DNA, 20 bp, synthetic construct
  source 1..20: mol_type = other DNA
  misc_feature 1..20: note = D-Lysine modified
tccacgctggccatcacgta

SEQ ID NO: 136 - DNA, 20 bp, synthetic construct
  source 1..20: mol_type = other DNA
tccacgctggccatcacgta

SEQ ID NO: 137 - DNA, 14 bp, synthetic construct
  source 1..14: mol_type = other DNA
tgggggttgtccac

SEQ ID NO: 138 - DNA, 15 bp, synthetic construct
  source 1..15: mol_type = other DNA
gcacacgtgggggtt

SEQ ID NO: 139 - DNA, 17 bp, synthetic construct
  source 1..17: mol_type = other DNA
  misc_feature 1..17: note = D-Lysine modified
acaacccccacgtgtgc

SEQ ID NO: 140 - DNA, 16 bp, synthetic construct
  source 1..16: mol_type = other DNA
ctgagccaggagaaac

SEQ ID NO: 141 - DNA, 17 bp, synthetic construct
  source 1..17: mol_type = other DNA
gtaaactgagccaggag

SEQ ID NO: 142 - DNA, 20 bp, synthetic construct
  source 1..20: mol_type = other DNA
atggcactagtaaactgagc

SEQ ID NO: 143 - DNA, 21 bp, synthetic construct
  source 1..21: mol_type = other DNA
atccatataactgaaagccaa

SEQ ID NO: 144 - DNA, 23 bp, synthetic construct
  source 1..23: mol_type = other DNA
accacatcatccatataactgaa

SEQ ID NO: 145 - DNA, 17 bp, synthetic construct
  source 1..17: mol_type = other DNA
  misc_feature 1..17: note = D-Lysine modified
ttgcccacaccgccggc

SEQ ID NO: 146 - DNA, 16 bp, synthetic construct
  source 1..16: mol_type = other DNA
  misc_feature 1..16: note = D-Lysine modified
tcttgcccacaccgcc

SEQ ID NO: 147 - DNA, 17 bp, synthetic construct
  source 1..17: mol_type = other DNA
  misc_feature 1..17: note = D-Lysine modified
tactcctcctggccggc

SEQ ID NO: 148 - DNA, 23 bp, synthetic construct
  source 1..23: mol_type = other DNA
cgtctccacagacacatactcca

SEQ ID NO: 149 - DNA, 23 bp, synthetic construct
  source 1..23: mol_type = other DNA
cgtctccacagacacatactcca

SEQ ID NO: 150 - DNA, 21 bp, synthetic construct
  source 1..21: mol_type = other DNA
gcctacgccaccagctccaac

SEQ ID NO: 151 - DNA, 21 bp, synthetic construct
  source 1..21: mol_type = other DNA
gcctacgccaccagctccaac

SEQ ID NO: 152 - DNA, 24 bp, synthetic construct
  source 1..24: mol_type = other DNA
ctacgccaccagctccaactacca

SEQ ID NO: 153 - DNA, 24 bp, synthetic construct
  source 1..24: mol_type = other DNA
ctacgccaccagctccaactacca

SEQ ID NO: 154 - DNA, 23 bp, synthetic construct
  source 1..23: mol_type = other DNA
tcttgcctacgccaccagctcca

SEQ ID NO: 155 - DNA, 23 bp, synthetic construct
  source 1..23: mol_type = other DNA
tgtactcctcttgacctgctgtg

SEQ ID NO: 156 - DNA, 23 bp, synthetic construct
  source 1..23: mol_type = other DNA
  misc_feature 1..23: note = D-Lysine modified
tgtactcctcttgacctgctgtg

SEQ ID NO: 157 - DNA, 23 bp, synthetic construct
  source 1..23: mol_type = other DNA
  misc_feature 1..23: note = Amino and carboxamido modified
ggcaaatcacatttatttcctac

SEQ ID NO: 158 - DNA, 23 bp, synthetic construct
  source 1..23: mol_type = other DNA
  misc_feature 1..23: note = D-Lysine modified
ggcaaatcacatttatttcctac

SEQ ID NO: 159 - DNA, 23 bp, synthetic construct
  source 1..23: mol_type = other DNA
  misc_feature 1..23: note = D-Lysine modified
tgtcttgtctttgctgatgtttc

SEQ ID NO: 160 - DNA, 23 bp, synthetic construct
  source 1..23: mol_type = other DNA
tgtcttgtctttgctgatgtttc

SEQ ID NO: 161 - DNA, 23 bp, synthetic construct
  source 1..23: mol_type = other DNA
  misc_feature 1..23: note = D-Lysine modified
tgtcttgtctttgctgatgtttc

SEQ ID NO: 162 - DNA, 21 bp, synthetic construct
  source 1..21: mol_type = other DNA
  misc_feature 1..21: note = D-Lysine modified
ctcttgacctgctgtgtcgag

SEQ ID NO: 163 - DNA, 21 bp, synthetic construct
  source 1..21: mol_type = other DNA
tcccaacaccacctgctccaa

SEQ ID NO: 164 - DNA, 25 bp, synthetic construct
  source 1..25: mol_type = other DNA
  misc_feature 1..25: note = D-Lysine modified
caacaccacctgctccaaccaccac

SEQ ID NO: 165 - DNA, 23 bp, synthetic construct
  source 1..23: mol_type = other DNA
cttttcccaacaccacctgctcc

SEQ ID NO: 166 - DNA, 25 bp, synthetic construct
  source 1..25: mol_type = other DNA
  misc_feature 1..25: note = D-Lysine modified
tgcgcttttcccaacaccacctgct

SEQ ID NO: 167 - DNA, 23 bp, synthetic construct
  source 1..23: mol_type = other DNA
ggcactgtactcttcttgtccag

SEQ ID NO: 168 - DNA, 21 bp, synthetic construct
  source 1..21: mol_type = other DNA
  misc_feature 1..21: note = D-Lysine modified
tctggtcttggctgaggtttc

SEQ ID NO: 169 - DNA, 23 bp, synthetic construct
  source 1..23: mol_type = other DNA
  misc_feature 1..23: note = Amino and carboxamido modified
ggcaaatcacacttgtttcccac

SEQ ID NO: 170 - DNA, 23 bp, synthetic construct
  source 1..23: mol_type = other DNA
  misc_feature 1..23: note = D-Lysine modified
ggcaaatcacacttgtttcccac

SEQ ID NO: 171 - DNA, 25 bp, synthetic construct
  source 1..25: mol_type = other DNA
  misc_feature 1..25: note = Amino and carboxamido modified
ttcttgtccagctgtatccagtatg

SEQ ID NO: 172 - DNA, 21 bp, synthetic construct
  source 1..21: mol_type = other DNA
  misc_feature 1..21: note = D-Lysine modified
agatcctctctctgaaatcac

SEQ ID NO: 173 - DNA, 24 bp, synthetic construct
  source 1..24: mol_type = other DNA
  misc_feature 1..24: note = D-Lysine modified
tctttctcctgctcagtgatttca

SEQ ID NO: 174 - DNA, 23 bp, synthetic construct
  source 1..23: mol_type = other DNA
  misc_feature 1..23: note = D-Lysine modified
aatgatgcacatcatggtggctg

SEQ ID NO: 175 - DNA, 23 bp, synthetic construct
  source 1..23: mol_type = other DNA
  misc_feature 1..23: note = D-Lysine modified
ggcactgtactcttcttgtccag

SEQ ID NO: 176 - DNA, 26 bp, synthetic construct
  source 1..26: mol_type = other DNA
  misc_feature 1..26: note = Amino modified
ttcatcaaccgcactctgtttatctc

SEQ ID NO: 177 - DNA, 25 bp, synthetic construct
  source 1..25: mol_type = other DNA
  misc_feature 1..25: note = Amino modified
tggcgacgacaatggacccaattat

SEQ ID NO: 178 - DNA, 30 bp, synthetic construct
  source 1..30: mol_type = other DNA
  misc_feature 1..30: note = Amino modified
agatgtagttagcaatcggtccttgttgta

SEQ ID NO: 179 - DNA, 30 bp, synthetic construct
  source 1..30: mol_type = other DNA
  misc_feature 1..30: note = Amino modified
gggtaattgaggtaacgtaggtatcaagat

SEQ ID NO: 180 - DNA, 27 bp, synthetic construct
  source 1..27: mol_type = other DNA
  misc_feature 1..27: note = Amino modified
tactatcgactgacatgaggcttgtgt

SEQ ID NO: 181 - DNA, 20 bp, synthetic construct
  source 1..20: mol_type = other DNA
  misc_feature 1..20: note = D-Lysine modified
agtccgacgatctggaattc

SEQ ID NO: 182 - DNA, 19 bp, synthetic construct
  source 1..19: mol_type = other DNA
  misc_feature 1..19: note = D-Lysine modified
actggagttcagacgtgtg

SEQ ID NO: 183 - DNA, 20 bp, synthetic construct
  source 1..20: mol_type = other DNA
  misc_feature 1..20: note = D-Lysine modified
ctcttccgatcagatcggaa

SEQ ID NO: 184 - DNA, 21 bp, synthetic construct
  source 1..21: mol_type = other DNA
  misc_feature 1..21: note = D-Lysine modified
ctcttccgatcagatcggaag

SEQ ID NO: 185 - DNA, 15 bp, synthetic construct
  source 1..15: mol_type = other DNA
  misc_feature 1..15: note = D-Lysine modified
agcgctccccgcacc

SEQ ID NO: 186 - DNA, 15 bp, synthetic construct
  source 1..15: mol_type = other DNA
  misc_feature 1..15: note = D-Lysine modified
agcgctccccgcacc

SEQ ID NO: 187 - DNA, 14 bp, synthetic construct
  source 1..14: mol_type = other DNA
  misc_feature 1..14: note = D-Lysine modified
ggggagcgctctgt

SEQ ID NO: 188 - DNA, 15 bp, synthetic construct
  source 1..15: mol_type = other DNA
  misc_feature 1..15: note = D-Lysine modified
agcgctccccgcacc

SEQ ID NO: 189 - DNA, 20 bp, synthetic construct
  source 1..20: mol_type = other DNA
  misc_feature 1..20: note = D-Lysine modified
tgcatacacactgcccgcct

SEQ ID NO: 190 - DNA, 360 bp, Homo sapiens
  source 1..360: mol_type = genomic DNA
tagaaattagatctcttacctaaactcttcataatgcttgctctgataggaaaatgagatctactgttttcctttacttactacacctcagatatatttcttcatgaagacctcacagtaaaaataggtgattttggtctagctacagtgaaatctcgatggagtgggtcccatcagtttgaacagttgtctggatccattttgtggatggtaagaattgaggctatttttccactgattaaatttttggccctgagatgctgctgagttactagaaagtcattgaaggtctcaactatagtattttcatagttcccagtattcacaaaaatcagtgttcttattttttatgtaaataga

SEQ ID NO: 191 - DNA, 480 bp, Homo sapiens
  source 1..480: mol_type = genomic DNA
tagagaaggcgtacatttgtccttccaaatgagctggcaagtgccgtgtcctggcacccaagcccatgccgtggctgctggtccccctgctgggccatgtctggcactgctttccagcatggtgagggctgaggtgacccttgtctctgtgttcttgtcccccccagcttgtggagcctcttacacccagtggagaagctcccaaccaagctctcttgaggatcttgaaggaaactgaattcaaaaagatcaaagtgctgggctccggtgcgttcggcacggtgtataaggtaaggtccctggcacaggcctctgggctgggccgcagggcctctcatggtctggtggggagcccagagtccttgcaagctgtatatttccatcatctactttactctttgtttcactgagtgtttgggaaactccagtgtttttcccaagttattgagaggaaatcttttataaccacagtaatcagtg

SEQ ID NO: 192 - DNA, 420 bp, Homo sapiens
  source 1..420: mol_type = genomic DNA
agcccaacagctgcagggctgcgggggcgtcacagcccccagcaatatcagccttaggtgcggctccacagccccagtgtccctcaccttcggggtgcatcgctggtaacatccacccagatcactgggcagcatgtggcaccatctcacaattgccagttaacgtcttccttctctctctgtcatagggactctggatcccagaaggtgagaaagttaaaattcccgtcgctatcaaggaattaagagaagcaacatctccgaaagccaacaaggaaatcctcgatgtgagtttctgctttgctgtgtgggggtccatggctctgaacctcaggcccaccttttctcatgtctggcagctgctctgctctagaccctgctcatctccacatcctaaatgttcactttctatgtctttcc

SEQ ID NO: 193 - DNA, 480 bp, Homo sapiens
  source 1..480: mol_type = genomic DNA
aaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctgg

SEQ ID NO: 194 - DNA, 420 bp, Homo sapiens
  source 1..420: mol_type = genomic DNA
ggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatc

SEQ ID NO: 195 - DNA, 239 bp, Homo sapiens
  source 1..239: mol_type = genomic DNA
ctgcaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacaggtgaaccccgtgaggctggcccgggagcccacgccgcacaggtggggccaggcc

SEQ ID NO: 196 - DNA, 770 bp, Homo sapiens
  source 1..770: mol_type = genomic DNA
ctgacatctacctctagttgtacttctgtcctctatttcaggtgttatgggtcaagcctgtttgactggcattattcatgattcctgtaccactcttgctctctctcactttgatctccatattccaggcttacacaggggtttcctcagaacgttgatggcagttgcaggtccatataaagggaccaaagcacattgtatcctcatctatagtcatgctgaaagtaggagaaagtgcatctttattatggcagagagaattttctgaactatttatggacaacagtcaaacaacaattctttgtacttttttttttccttagtctttctttgaagcagcaagtatgatgagcaagctttctcacaagcatttggttttaaattatggagtatgtgtctgtggagacgagagtaagtaaaactacaggctttctaatgcctttctcagagcatctgtttttgtttatatagaaaattcagtttcaggatcacagctaggtgtcagtgtaaactataatttaacaggagttaagtatttttgaaactgaaaacactgtaggactattcagttatatcttgtgaaaaaggaaagcaatgaagttaaaagtagaaggttacaatgcccaaacaatagagtattatagtaaacaaatgtctataaaacattttgtgttcatgatagcaaaagagattatggcaggttcaacataacattggaataactggccttttcagtacaaacttatctggaattatgaagacaaagcata

SEQ ID NO: 197 - DNA, 240 bp, Homo sapiens
  source 1..240: mol_type = genomic DNA
ggtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtg

SEQ ID NO: 198 - DNA, 240 bp, Homo sapiens
  source 1..240: mol_type = genomic DNA
cttctcaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattataggtgggtttaaattgaatataataagctgacattaaggagtaattatagttttt

SEQ ID NO: 199 - DNA, 300 bp, Homo sapiens
  source 1..300: mol_type = genomic DNA
gtgctataacttttttttctttcccagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacaggtaagtaacactgaaataaatacagatctgttttctgcaaaatcataactgttatgtcatttaatatatcagtttttctctcaattatgctatactaggaaataaaacaatat

SEQ ID NO: 200 - DNA, 180 bp, Homo sapiens
  source 1..180: mol_type = genomic DNA
aatgcaacagactttaaagaagttgtgttttacaatgcagagagtggaggatgctttttatacattggtgagggagatccgacaatacagattgaaaaaaatcagcaaagaagaaaagactcctggctgtgtgaaaattaaaaaatgcattataatgtaatctggtaagtttaagttcag

SEQ ID NO: 201 - DNA, 480 bp, Homo sapiens
  source 1..480: mol_type = genomic DNA
gtgtttttgcgttctctagtcactttaagaaccaaatggaaggtcacactagggttttcatttccattgattatagaaagctttaaagtactgtagatgtggctcgccaattaaccctgattactggtttccaacaggttcttgctggtgtgaaatgactgagtacaaactggtggtggttggagcaggtggtgttgggaaaagcgcactgacaatccagctaatccagaaccactttgtagatgaatatgatcccaccatagaggtgaggcccagtggtagcccgctgacctgatcctgtctctcacttgtcggatcatctttacccatattctgtattaaaggaataagaggagagaaagtaaaaagttattttgggtatacattcagttatgcaataagcttaacgtgtttatagagaacagttcatttttattagctgctgaagtttctaaaacctgtccagtttttaacagttct

SEQ ID NO: 202 - DNA, 480 bp, Homo sapiens
  source 1..480: mol_type = genomic DNA
tgggcttgaatagttagatgcttatttaaccttggcaatagcattgcattccctgtggtttttaataaaaattgaacttccctccctccctgcccccttaccctccacacccccaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacaggtactaggagcattattttctctgaaaggatgatctttgtgttctgaatctttatggggaaatgaggttaccacactagggaagatagagctttttaattatgggaagagttggttttaggttgtttgacattgagaatctagggtaattactgaaagttaatactggaatttattttacataat

SEQ ID NO: 203 - DNA, 420 bp, Homo sapiens
  source 1..420: mol_type = genomic DNA
tggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaag

SEQ ID NO: 204 - DNA, 360 bp, Homo sapiens
  source 1..360: mol_type = genomic DNA
tgtaaaatttattgaaaatgtatttgctttttctgtaaatcatctgtgaatccagaggggaaaaatatgacaaagaaagctatataagatattattttattttacagagtaacagactagctagagacaatgaattaagggaaaatgacaaagaacagctcaaagcaatttctacacgagatcctctctctgaaatcactgagcaggagaaagattttctatggagtcacaggtaagtgctaaaatggagattctctgtttctttttctttattacagaaaaaataactgaatttggctgatctcagcatgtttttaccatacctattggaataaataaagcagaatttacatgattttt

SEQ ID NO: 205 - DNA, 300 bp, Homo sapiens
  source 1..300: mol_type = genomic DNA
tagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataact

SEQ ID NO: 206 - DNA, 490 bp, Homo sapiens
  source 1..490: mol_type = genomic DNA
gttgtaaatctttgtaacacttcaaaaagctatattgtatttatattttaaaataaatttcagggtaaaataataataaagcaaaggtacctagtaaagtttttaactattttaaaggcttgaagagtgtcgaattatgtcctctgcaaaaaggccactgtggttgaattgggagaacccagacatcatgtcagagttactgtttcagaacaatgagatcatctttaaaaatggggatggtaaggaagagtattaatgagcttatgatgcatgaatttagctatctttttatacacaggatatttatgaaccatgaaaactactgaaagccatttaaggaatatacacatgtgataaaatatgtaatatttatcagatgtcttgacctttgaaatatgcatgtataatcaatgaaaagaaaagaagtactaggtttagatcagaagtcctgaaatcagttttttgttttttctttttcctgttccctgcc